(19) United States
(12) Patent Application Publication
(10) Pub. No.: US 2022/0185867 A1
(43) Pub. Date: Jun. 16, 2022

(54) MODIFIED HEMOGLOBIN MOLECULES AND USES THEREOF

(71) Applicant: University of Pittsburgh - Of the Commonwealth System of Higher Education, Pittsburgh, PA (US)

(72) Inventors: Anthony W. DeMartino, Pittsburgh, PA (US); Jason J. Rose, Fox Chapel, PA (US); Qinzi Xu, Wexford, PA (US); Mark T. Gladwin, Pittsburgh, PA (US); Jesus Tejero Bravo, Pittsburgh, PA (US)

(73) Assignee: University of Pittsburgh - Of the Commonwealth System of Higher Education, Pittsburgh, PA (US)

(21) Appl. No.: 17/601,039

(22) PCT Filed: Apr. 2, 2020
(86) PCT No.: PCT/US20/26440

§ 371 Date: Oct. 1, 2021

Related U.S. Application Data
(60) Provisional application No. 62/828,269, filed on Apr. 2, 2019.

(51) Int. Cl. C07K 14/805

(52) U.S. Cl. CPC C07K 14/805; A61K 38/00

(57) ABSTRACT

Compositions that include a globin, such as hemoglobin, in a relaxed state are described. Globin molecules in a relaxed state (R state) have a higher binding affinity for carbon monoxide and oxygen than globin molecules in a tense state (T state). Hemoglobin in a relaxed state can be, for example, hemoglobin that is substantially free of 2,3-diphosphoglycerate or hemoglobin that includes a β-Cys93 that is covalently modified to inhibit one or both salt bridges between β-Asp94, β-His146 and α-Lys40. Methods for using these compositions, such as for treating carbon monoxide poisoning, and methods for producing these compositions, are also disclosed.

### CROSS REFERENCE TO RELATED APPLICATIONS

This application claims the benefit of U.S. Provisional Application No. 62/828,269, filed Apr. 2, 2019, which is herein incorporated by reference in its entirety.

### STATEMENT OF GOVERNMENT SUPPORT

This invention was made with government support under grant numbers HL098032; HL007563; HL110849; HL103455; HL136857 and HL125886, awarded by the National Institutes of Health. The government has certain rights in the invention.

### FIELD

This disclosure concerns compositions including globin in a relaxed state, such as hemoglobin that is 2,3-diphosphoglyerate-free and/or a hemoglobin including a β-Cys93 residue that is covalently modified. This disclosure further concerns methods of treating carboxyhemoglobinemia and a process for producing a hemoglobin including a modified β-Cys93 residue.

### BACKGROUND

Inhalation exposure to carbon monoxide represents a major cause of environmental poisoning. Individuals can be exposed to carbon monoxide in the air under a variety of circumstances, such as house fires, use of generators or outdoor barbeque grills used inside the house, or during suicide attempts in closed spaces. Carbon monoxide binds to hemoglobin and to hemoproteins in cells, in particular, the enzymes of the respiratory transport chain. The accumulation of carbon monoxide bound to hemoglobin and other hemoproteins impairs oxygen delivery and oxygen utilization for oxidative phosphorylation. This ultimately results in severe hypoxic and ischemic injury to vital organs such as the brain and the heart. Individuals who accumulate greater than 5-10% carbon carboxyhemoglobin in their blood, as well as individuals with chronic low level poisoning, are at risk for brain injury and neurocognitive dysfunction. Patients with very high carboxyhemoglobin levels typically suffer from irreversible brain injury, respiratory failure, cardiovascular collapse and/or death.

Despite the availability of methods to rapidly diagnose carbon monoxide poisoning with standard arterial and venous blood gas analysis and co-oximetry, and despite an awareness of risk factors for carbon monoxide poisoning, there are no available antidotes for this toxic exposure. The current therapy is to give 100% oxygen by face mask, and when possible, to expose patients to hyperbaric oxygen. Hyperbaric oxygen therapy increases the rate of release of the carbon monoxide from hemoglobin and from tissues, and accelerates the natural clearance of carbon monoxide. However, this therapy has only a modest effect on carbon monoxide clearance rates and based on the complexity of hyperbaric oxygen facilities, this therapy is not available in the field and is often associated with significant treatment delays and transportation costs. Thus, a need exists for an effective, rapid and readily available therapy to treat carbon monoxide poisoning, also known as carboxyhemoglobinemia.

### SUMMARY

Described herein are isolated, modified globin molecules that bind and remove carbon monoxide (CO) from CO-poisoned hemoglobin in the bloodstream and from CO-poisoned cytochrome c oxidase in the mitochondria, thereby functioning as CO scavengers. Also described are methods of producing the modified globin molecules, methods of removing carbon monoxide from hemoglobin in blood or tissues, methods of removing carbon monoxide from mitochondria in tissue, and methods for treating carbon monoxide poisoning (also known as “carboxyhemoglobinemia”) with the modified globin molecules.

Provided herein is a composition that includes a globin in a relaxed state. In some embodiments, the globin is myoglobin or hemoglobin. In some examples, the hemoglobin is substantially free of 2,3-diphosphoglycerate. In some examples, the globin is a modified myoglobin or hemoglobin. In particular examples, the globin is a modified hemoglobin that includes a β-Cys93 that is covalently modified to inhibit one or both salt bridges between β-Asp94, β-Hys146 and α-Lys40. Isolated hemoglobin molecules that include a β-Cys93 covalently modified to inhibit one or both salt bridges between β-Asp94, β-His146 and α-Lys40 is further provided.

Also provided are methods of treating carboxyhemoglobinemia in a subject. In some embodiments, the method includes selecting a subject with carboxyhemoglobinemia; and administering to the subject a therapeutically effective amount of a composition or isolated hemoglobin disclosed herein.

Further provided is a method of removing carbon monoxide from hemoglobin in blood or animal tissue. In some embodiments, the method includes contacting the blood or animal tissue with a composition or isolated hemoglobin disclosed herein.

Methods of producing the modified globin molecules disclosed herein are also provided. In some embodiments, the method includes isolating hemoglobin from whole blood, packed red blood cells, or a combination thereof; reacting the hemoglobin with a reactant, such as a reactant having a structure satisfying any one or more of Formulas I-V, to break disulfide bridges and form hemoglobin which is covalently modified at β-Cys93; and isolating the hemoglobin which is covalently modified at β-Cys93.

The foregoing and other objects and features of the present disclosure will become more apparent from the following detailed description of several embodiments which proceeds with reference to the accompanying figures.

### BRIEF DESCRIPTION OF THE FIGURES

FIG. 1 depicts modified hemoglobin (Hb) molecules. Formation of critical salt bridges between β-Asp94 and β-His146 as well as β-His146 and α-Lys40 helps generate the T state. Modifying β-Cys93 (with R′ groups listed above) interrupts these salt bridges, allowing for even non-ligated Hb (i.e., no O₂ or CO bound) to remain in the R state. This form (dashed box) allows for tighter bonding of CO and more efficient scavenging. Each subunit contains one heme each for binding CO, though not depicted in this representation.

FIG. 2 is a graph showing decay of the hemoglobin-CO species under therapeutic treatments. Half-life values of HbCO in room air (320 minutes), 100% normobaric oxygen (74 minutes), and 100% hyperbaric oxygen (HBO2; 20 minutes), from Rose et al. (Am J Respir Crit Care Med 195(5): 596-606, 2017).

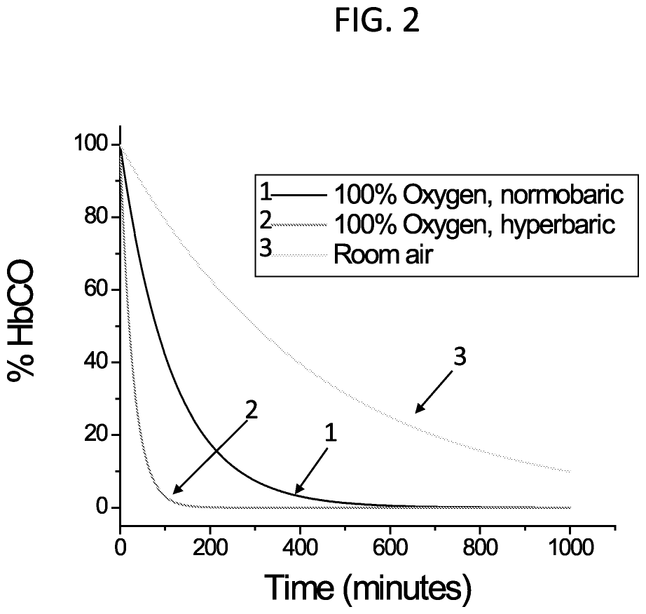

FIG. 3 is a graph showing the in vivo binding of CO from hemoglobin to recombinant neuroglobin in a mouse model for moderate CO poisoning.

FIG. 4 is a chart of mitochondrial respiration inhibited by CO reversed with the addition of stripped Hb (StHb).

FIG. 5 is a flow diagram of the steps of a method for the preparation of a deoxygenated globin molecule.

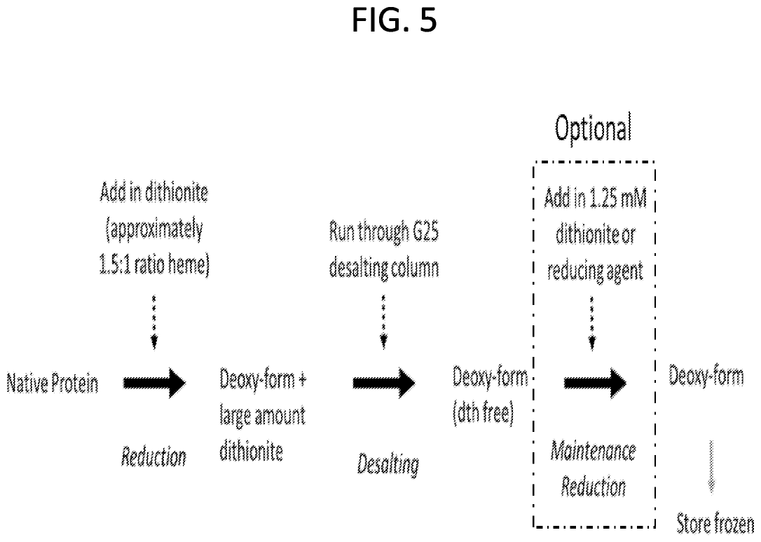

FIG. 6 is a flow diagram of the steps of a method for use of specifically modified, 2,3-DPG free hemoglobin to treat carbon monoxide poisoning.

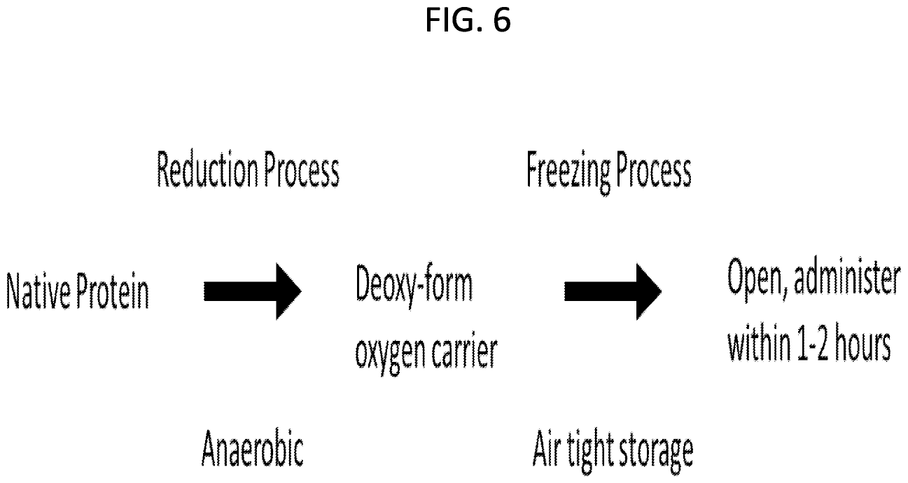

FIG. 7 is a graph showing 2,3-DPG levels in relation to hemoglobin concentration for fresh mouse isolated hemoglobin, commercially available hemoglobin (Sigma Aldrich), stripped hemoglobin and stripped hemoglobin further treated with NaCl, dithionite, and through a G25 separation column.

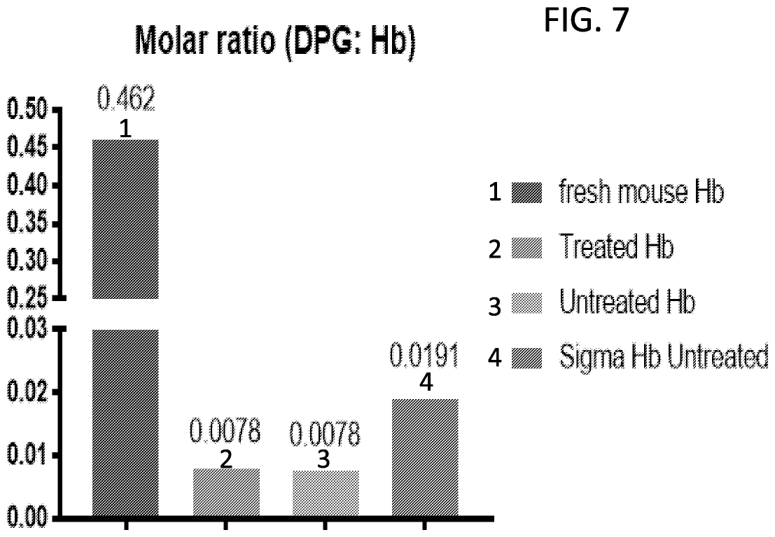

FIGS. 8A-8C are a set of graphs showing the results of an in vitro study of carbon monoxide saturated red blood cells (RBC) (as represented by amount of RBC encapsulated hemoglobin bound to CO (HbCO)) combined with StHb and NEMHb over time. (FIGS. 8A-8B) NEMHb binds to CO more effectively than StHb as represented by RBC encapsulated Hb isolated from RBC pellet (FIG. 8A) and by measuring supernatant CO bound specified hemoglobin molecules (FIG. 8B). (FIG. 8C) At equilibrium after some period of time, the HbCO levels of RBC encapsulated hemoglobin are lower in further modified 2,3-DPG reduced hemoglobin.

FIG. 9A is a graph showing binding of StHb, NEM-Hb and myoglobin (Mb) to CO in CO poisoned animals. StHb and NEM-Hb exhibit significantly greater levels of CO binding compared to Mb. FIG. 9B is a graph showing the reduction in HbCO after infusion of PBS, StHb, NEM-Hb and Mb. NEM-Hb and StHb infusion reduces the HbCO level significantly more effectively than control PBS and similar to myoglobin.

FIG. 10 shows that mice exposed to severe CO poisoning develop hypotension and die. In PBS, there is 100% mortality in this model. Myoglobin (Mb), NEM-Hb and stripped hemoglobin (StHb) reverse cardiovascular collapse and hypotension.

FIG. 11 shows a Kaplan-Meier survival analysis of mice exposed to severe CO poisoning for up to 40 minutes. In PBS-treated animals, there is 0% survival in this model. In contrast, administration of Mb, NEM-Hb or StHb increases survival.

FIG. 12 is a graph showing the in vivo binding of CO from HbCO to hemoglobin, myoglobin and NEM-Hb in a mouse model for CO poisoning over time.

FIG. 13 is a graph demonstrating the reduction in HbCO immediately after infusion of hemoproteins or PBS. HbCO was significantly reduced by infusion of StHb, NEM-Hb and Mb, relative to PBS.

FIG. 14 is a graph showing the effects of moderate CO poisoning on blood pressure reversed with the addition of Mb, StHb and NEM-Hb in mice.

FIG. 15 is a flow diagram of the setup for mitochondrial respiration studies. After addition of ADP/succinate, mitochondria respire to the desired O₂ concentration, then the system is reoxygenated, and mitochondria respire to the desired level O₂ again. CO is then infused, the system is reoxygenated, and rates of respiration are compared. After respiration to 0% O₂, stripped hemoglobin is infused, the system is reoxygenated and rates are compared.

FIGS. 16A-16C are graphs showing the effects of CO on mitochondrial respiration and the reversal of these effects with stripped hemoglobin. (FIG. 16A) Representative raw data of Clark electrode chamber demonstrating the setup for the CO exposure followed by oxy-stripped Hb treatment experiment. (FIG. 16B) Representative raw data of Clark electrode chamber demonstrating the setup for the CO exposure only experiment. (FIG. 16C) Respiration rates compared to initial reoxygenation step rate.

FIG. 17 is a set of graphs showing blood chemistries in mice after treatment with normal saline control (NS); 4000 mg/kg albumin control; 100 mM N-acetyl cysteine (NAC) control; 4 mM NEM-Hb+40 mM NAC (1600 mg/kg NEM-Hb, regular dose); 4 mM stripped Hb+40 mM NAC (1600 mg/kg stripped Hb, regular dose); 10 mM NEM-Hb+100 mM NAC (4000 mg/kg NEM-Hb, medium dose); 10 mM stripped Hb+100mM NAC (4000 mg/kg stripped Hb, medium dose).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Mar. 27, 2020, 21.5 KB, which is incorporated by reference herein. In the accompanying sequence listing:

SEQ ID NOs: 1 and 2 are the amino acid sequences of the human hemoglobin alpha and beta subunits, respectively.

SEQ ID NOs: 3 and 4 are the amino acid sequences of the canine hemoglobin alpha and beta subunits, respectively.

SEQ ID NOs: 5 and 6 are the amino acid sequences of the porcine hemoglobin alpha and beta subunits, respectively.

SEQ ID NOs: 7 and 8 are the amino acid sequences of the equine hemoglobin alpha and beta subunits, respectively.

SEQ ID NOs: 9 and 10 are the amino acid sequences of the bovine hemoglobin alpha and beta subunits, respectively.

SEQ ID NOs: 11 and 12 are the amino acid sequences of the murine hemoglobin alpha and beta subunits, respectively.

SEQ ID NOs: 13 and 14 are the amino acid sequences of the feline hemoglobin alpha and beta subunits, respectively.

SEQ ID NOs: 15 and 16 are the amino acid sequences of the Rhesus macaque hemoglobin alpha and beta subunits, respectively.

SEQ ID NO: 17 is a nucleic acid sequence of human hemoglobin.

### DETAILED DESCRIPTION OF SEVERAL EMBODIMENTS

Disclosed herein are isolated, modified globin molecules that function as carbon monoxide scavengers by binding and removing carbon monoxide from hemoglobin in the bloodstream and cytochrome c oxidase in the mitochondria. Also disclosed are methods of producing the modified globin molecules, and methods for treating carbon monoxide poisoning with the modified globin molecules. There is a component of CO poisoning related to locally elevated levels of nitric oxide (NO), and the disclosed molecules also treat this aspect of the disease (Thom et al., Toxicol Appl Pharmacol 1994;128:105-110; Thom et al., Chem Res. Toxicol 1997; 10:1023-1031; Rose et al., Am J Respir Crit Care Med 2017; 195(5):596-606). The data disclosed herein demonstrates that these agents (specifically modified 2,3-DPG free hemoglobin) can be used, for example, in methods of removing carbon monoxide from hemoglobin in blood or tissue, removing carbon monoxide from mitochondria in tissue, and in methods of treating carboxyhemoglobinemia. Myoglobin and hemoglobin are five-coordinated heme proteins that only have one histidine permanently bound to the heme. Myoglobin has an affinity for CO that is sixty times that of O₂ (Nelson L S, Lewin N A, Howland M A, Hoffman R S, Goldfrank L R, Flomenbaum N E. (2011). “Carbon Monoxide”. Goldfrank's Toxicologic Emergencies (9th ed.). New York: McGraw-Hill. pp. 1658-1670). The reaction of the iron atom from a heme group can be depicted as follows:

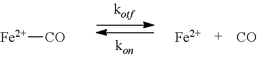

where kₒₙ and k_{off} are the rate constants of CO binding and dissociation, respectively.

Non-CO bound Hb can act as an additional target for CO, as reduced Hb in the presence of CO will act as a reservoir for CO binding. Modified globin molecules will act in a similar manner as naturally occurring compounds. Additionally, these agents can be given already bound with oxygen, increasing oxygen delivery to tissue while binding up CO.

Hemoglobin oxygen release to tissues is controlled by erythrocytic 2,3-diphosphoglycerate (2,3-DPG) such that an increase in the concentration of 2,3-DPG decreases oxygen affinity and vice versa. The increased oxygen affinity of blood stored in acid-citrate-dextrose solution has been shown to be due to the decrease in the concentration of 2,3-DPG that occurs during storage. 2,3-DPG stabilizes the tense, deoxy form of hemoglobin and so reduces oxygen affinity.

The central cavity of relaxed oxyhemoglobin is smaller and is therefore unable to accommodate 2,3-DPG. The 2,3-DPG also binds non-specifically to the N-terminal amino-groups of the 8-chains of both oxy and deoxyhemoglobin.

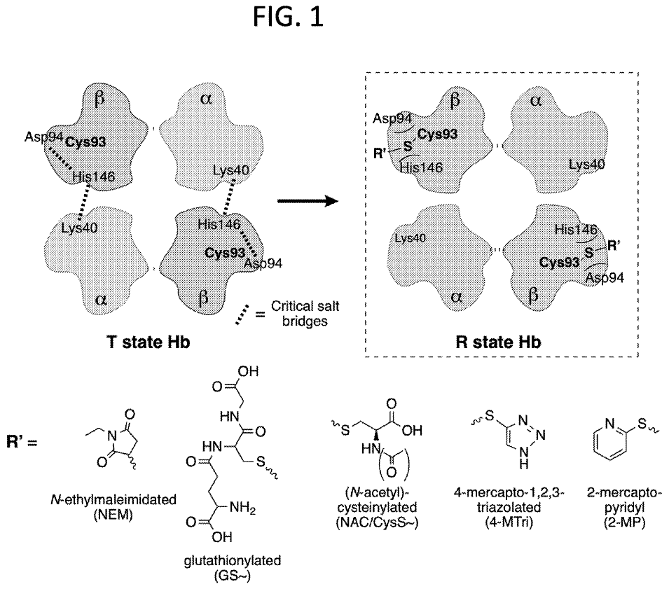

The Hb tetramer exists in two conformations, the “relaxed” state (R state) and “tense” state (T-state) (FIG. 1). The conformation of the T-state has lower affinity for oxygen, which allows for oxygen delivery; the R state has higher affinity for oxygen allowing for binding to the tetramer in the lung.

Disclosed herein are compositions comprising a globin, such as hemoglobin or myoglobin, in a relaxed state, wherein at least 85% of the globin is in the relaxed state.

In some embodiments, the globin is hemoglobin. In specific non-limiting examples, the hemoglobin is substantially free of 2,3-DPG. In specific non-limiting examples, the hemoglobin includes a β-Cys93 that is covalently modified to inhibit one or both salt bridges between (1) (3-Asp94 and β-Hys146; and (2) β-Hys146 and α-Lys40. Also disclosed are methods for producing these molecules.

In further embodiments, methods of using relaxed state globin molecules and compositions thereof are disclosed.

### I. Abbreviations

2,3-DPG 2,3-diphosphoglycerate

CO carbon monoxide

Hb hemoglobin

HbCO carboxyhemoglobin

Mb myoglobin

NEMHb N-ethylmaleimide hemoglobin

NO nitric oxide

StHb stripped hemoglobin

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting. The term “about” means within five percent, unless otherwise stated.

Certain functional group terms herein include a symbol “-” which is used to show how the defined functional group attaches to, or within, the compound to which it is bound. Also, a dashed bond (i.e., “ [symbol] ”) as used in certain formulas described herein indicates an optional bond (that is, a bond that may or may not be present). A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

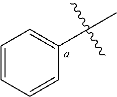 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

In order to facilitate review of the various embodiments of the disclosure, the following explanations of specific terms are provided:

Acyl Halide: —C(O)X, wherein X is a halogen, such as Br, F, I, or Cl.

Administration: To provide or give a subject an agent, such as a therapeutic agent (e.g., an oxygen carrier such as a modified globin), by any effective route. Exemplary routes of administration include, but are not limited to, injection or infusion (such as subcutaneous, intramuscular, intradermal, intraperitoneal, intrathecal, intravenous, intracerebroventricular, intrastriatal, intracranial and into the spinal cord), oral, intraductal, sublingual, rectal, transdermal, intranasal, vaginal and inhalation routes.

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Aliphatic-aromatic: An aromatic group that is or can be coupled to a compound disclosed herein, wherein the aromatic group is or becomes coupled through an aliphatic group.

Aliphatic-aryl: An aryl group that is or can be coupled to a compound disclosed herein, wherein the aryl group is or becomes coupled through an aliphatic group.

Aliphatic-heteroaryl: A heteroaryl group that is or can be coupled to a compound disclosed herein, wherein the heteroaryl group is or becomes coupled through an aliphatic group.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Antidote: An agent that neutralizes or counteracts the effects of a poison.

Amide: —C(O)NR^{a}R^{b} or —NR^{a}C(O)R^{b} wherein each of R^{a} and R^{b} independently is selected from hydrogen, aliphatic, heteroaliphatic, haloaliphatic, haloheteroaliphatic, aromatic, or an organic functional group.

Amino: —NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

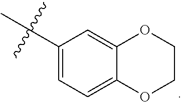 [SMILES: *c1ccc2c(c1)OCCO2]

However, in certain examples, context or express disclosure may indicate that the point of attachment is through a non-aromatic portion of the condensed ring system. For example,

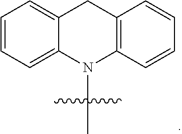 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic.

Azo: —N═NR^{a} wherein R^{a} is hydrogen, aliphatic, heteroaliphatic, haloaliphatic, haloheteroaliphatic, aromatic, or an organic functional group.

Carbamate: —C(O)NR^{a}R^{b}, wherein each of R^{a} and R^{b} independently is selected from hydrogen, aliphatic, heteroaliphatic, haloaliphatic, haloheteroaliphatic, aromatic, or an organic functional group.

Carboxyl: —C(O)OH.

Carboxylate: —C(O)O⁻ or salts thereof, wherein the negative charge of the carboxylate group may be balanced with an M⁺ counterion, wherein M³⁰ may be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Cyano: —CN.

Carbon monoxide (CO): A colorless, odorless and tasteless gas that is toxic to humans and animals when encountered at sufficiently high concentrations. CO is also produced during normal animal metabolism at low levels.

Carboxyhemoglobin (HbCO or CO-Hb): A stable complex of carbon monoxide (CO) and hemoglobin (Hb) that forms in red blood cells when CO is inhaled or produced during normal metabolism.

Carboxyhemoglobinemia or carbon monoxide poisoning: A condition resulting from the presence of excessive amounts of carbon monoxide in the blood. Typically, exposure to CO of 100 parts per million (ppm) or greater is sufficient to cause carboxyhemoglobinemia. Symptoms of mild acute CO poisoning include lightheadedness, confusion, headaches, vertigo, and flu-like effects; larger exposures can lead to significant toxicity of the central nervous system and heart, and even death. Following acute poisoning, long-term sequelae often occur. Carbon monoxide can also have severe effects on the fetus of a pregnant woman. Chronic exposure to low levels of carbon monoxide can lead to depression, confusion, and memory loss. Carbon monoxide mainly causes adverse effects in humans by combining with hemoglobin to form carboxyhemoglobin (HbCO) in the blood. This prevents oxygen binding to hemoglobin, reducing the oxygen-carrying capacity of the blood, leading to hypoxia. Additionally, myoglobin and mitochondrial cytochrome c oxidase are thought to be adversely affected. Carboxyhemoglobin can revert to hemoglobin, but the recovery takes time because the HbCO complex is fairly stable. Current methods of treatment for CO poisoning including administering 100% oxygen or providing hyperbaric oxygen therapy.

Contacting: Placement in direct physical association; includes both in solid and liquid form. When used in the context of an in vivo method, “contacting” also includes administering.

Cyanide poisoning: A type of poisoning that results from exposure to some forms of cyanide, such as hydrogen cyanide gas and cyanide salt. Cyanide poisoning can occur from inhaling smoke from a house fire, exposure to metal polishing, particular insecticides and certain seeds (such as apple seeds). Early symptoms of cyanide poisoning include headache, dizziness, rapid heart rate, shortness of breath and vomiting. Later symptoms include seizures, slow heart rate, low blood pressure, loss of consciousness and cardiac arrest.

Cytoglobin: A globin molecule that is ubiquitously expressed in all tissues. Cytoglobin is a hexacoordinate hemoglobin that has been reported to facilitate diffusion of oxygen through tissues, to reduce nitrite to nitric oxide, and to play a cytoprotective role in hypoxic conditions and under oxidative stress conditions.

Disulfide: —SSR^{a}, wherein R^{a} is selected from hydrogen, aliphatic, heteroaliphatic, haloaliphatic, haloheteroaliphatic, aromatic, or an organic functional group.

Dithiocarboxylic: —C(S)SR^{a} wherein R^{a} is selected from hydrogen, aliphatic, heteroaliphatic, haloaliphatic, haloheteroaliphatic, aromatic, or an organic functional group.

Ester: —C(O)OR^{a} or —OC(O)R^{a}, wherein R^{a} is selected from aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Globin: A heme-containing protein involved in the binding and/or transport of oxygen. Globins include, for example, hemoglobin, myoglobin, neuroglobin and cytoglobin. Globin molecules include hemoglobin (Hb) originating from, for example, humans, bovines, or other living organisms; concentrated red blood cells; and myoglobin originating from, for example, humans, bovines, or other living organisms.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a compound disclosed herein, wherein the aryl group is or becomes coupled through a haloaliphatic group.

Haloaliphatic-heteroaryl: A heteroaryl group that is or can be coupled to a compound disclosed herein, wherein the heteroaryl group is or becomes coupled through a haloaliphatic group. Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Hemocyanin: A type of protein that transports oxygen throughout the body of some invertebrate animals. Hemocyanins are metalloproteins that contain two copper atoms that reversibly bind a single oxygen molecule. Hemocyanins are found only in the phylum Mollusca and the phylum Arthropoda.

Hemoglobin (Hb): The iron-containing oxygen-transport metalloprotein in red blood cells of vertebrates and other animals. In humans, the hemoglobin molecule is an assembly of four globular protein subunits. Each subunit is composed of a protein chain tightly associated with a non-protein heme group. Each protein chain arranges into a set of alpha-helix structural segments connected together in a globin fold arrangement, so called because this arrangement is the same folding motif used in other heme/globin proteins. This folding pattern contains a pocket which strongly binds the heme group. In the context of the present disclosure, a globin, such as hemoglobin, in the “tense state” is a globin in the “T state” and a globin, such as hemoglobin, in the “relaxed state” is a globin in the R state (see FIG. 1). Salt bridges between β-Asp94 and β-His146, and between β-His146 and α-Lys40, help generate the T state of hemoglobin. It is disclosed herein that modification of β-Cys93 with particular reactants (such as NEM) disrupts these salt bridges converting Hb to the R state. Hb in the R state possesses increased affinity towards oxygen and CO compared to Hb in the T-state. As used herein, “stripped hemoglobin” or “StHb” refers to hemoglobin that lacks or substantially lacks 2,3-DPG. StHb is also found in the R-state.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteroaliphatic group, such as when the heteroaliphatic group is a heterocyclic group.

Heteroaliphatic-aryl: An aryl group that is or can be coupled to a compound disclosed herein, wherein the aryl group is or becomes coupled through a heteroaliphatic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom. Heterologous: A heterologous protein or polypeptide refers to a protein or polypeptide derived from a different source or species.

Hydrogen sulfide poisoning: A type of poisoning resulting from excess exposure to hydrogen sulfide (H₂S). H₂S binds iron in the mitochondrial cytochrome enzymes and prevents cellular respiration. Exposure to lower concentrations of H25 can cause eye irritation, sore throat, coughing, nausea, shortness of breath, pulmonary edema, fatigue, loss of appetite, headaches, irritability, poor memory and dizziness. Higher levels of exposure can cause immediate collapse, inability to breath and death.

Isolated: An “isolated” biological component (such as a globin, nucleic acid molecule, protein, or cell) has been substantially separated or purified away from other biological components in the cell, blood or tissue of the organism, or the organism itself, in which the component naturally occurs, such as other chromosomal and extra-chromosomal DNA and RNA, proteins and cells. Nucleic acid molecules and proteins, such as globins, that have been “isolated” include those purified by standard purification methods. The term also embraces nucleic acid molecules and proteins, such as a globin, prepared by recombinant expression in a host cell as well as chemically synthesized nucleic acid molecules and proteins, such as a globin.

Ketone: —C(O)R^{a}, wherein R^{a} is selected from aliphatic, heteroaliphatic, haloaliphatic, haloheteroaliphatic, aromatic, or an organic functional group.

Methemoglobin: The oxidized form of hemoglobin in which the iron in the heme component has been oxidized from the ferrous (+2) to the ferric (+3) state. This renders the hemoglobin molecule incapable of effectively transporting and releasing oxygen to the tissues. Normally, there is about 1% of total hemoglobin in the methemoglobin form.

Myoglobin (Mb): A member of the globin family of proteins. Myoglobin is an iron- and oxygen-binding protein found in the muscle tissue of all vertebrates and nearly all mammals. In humans, myoglobin is only found in the bloodstream after muscle injury. Unlike hemoglobin, myoglobin contains only one binding site for oxygen (on the one heme group of the protein), but its affinity for oxygen is greater than the affinity of hemoglobin for oxygen.

Neuroglobin (Ngb): A member of the globin family of proteins. The physiological function of neuroglobin is currently unknown, but is thought to provide protection under hypoxic or ischemic conditions. Neuroglobin is expressed in the central and peripheral nervous system, cerebral spinal fluid, retina and endocrine tissues.

Organic functional group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol;

sulfonyl (or sulfonate); oxime; ester; thiocyanate; thioketone; thiocarboxylic acid; thioester; dithiocarboxylic acid or ester; phosphonate; phosphate; silyl ether; sulfinyl; thial; or combinations thereof.

Oxidizing agent: A substance that is capable of accepting an electron from another substance (also referred to as “oxidizing” a substance). An oxidizing agent gains electrons and is reduced in a chemical reaction. An oxidizing agent is also known as an “electron acceptor.”

Oxime: —CRa═NOH, wherein R^{a} is hydrogen, aliphatic, heteroaliphatic, haloaliphatic, haloheteroaliphatic, aromatic, or an organic functional group.

Oxygen carrier: Molecules or compounds that are capable of binding, transporting and releasing oxygen in the body. Oxygen carriers include natural proteins, such as hemoglobin, myoglobin and hemocyanin, as well as artificial oxygen carriers, including hemoglobin-based oxygen carriers (HBOCs), perfluorocarbons (PFCs), liposome-encapsulated hemoglobin and porphyrin metal complexes.

Peptide or Polypeptide: A polymer in which the monomers are amino acid residues which are joined together through amide bonds. When the amino acids are alpha-amino acids, either the L-optical isomer or the D-optical isomer can be used, the L-isomers being preferred. The terms “peptide,” “polypeptide” or “protein” as used herein are intended to encompass any amino acid sequence and include modified sequences, including modified globin proteins. The terms “peptide” and “polypeptide” are specifically intended to cover naturally occurring proteins, as well as those which are recombinantly or synthetically produced. A peptide can include common terminal amino acid modifications such as carbamylated (e.g., —CO₂ addition to amines), alkylation (e.g., methylation leading to alkylamine formation) or organic carbamation (such as functionalizing an amine group with a protecting group leading to a carbamate, wherein protecting groups can be, but are not limited to, tert-butoxycarbony (BOC) or fluorenylmethyloxycarbonyl (Fmoc)), carbamoylation (e.g., addition of a —C(O)NH₂ group), or combinations thereof.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartic acid; or (d) a residue having a bulky side chain, for example, phenylalanine, is substituted for (or by) one not having a side chain, for example, glycine.

Peroxy: —O—P^{a} wherein R^{a} is hydrogen, aliphatic, heteroaliphatic, haloaliphatic, haloheteroaliphatic, aromatic, or an organic functional group.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, Pa., 21s^{t} Edition (2005), describes compositions and formulations suitable for pharmaceutical delivery of the globin molecules and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phosphate: —O—P(O)(ORa)₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M³⁰ independently can be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Porphyrin: An organic compound containing four pyrrole rings, functioning as a metal-binding cofactor in hemoglobin, chlorophyll and certain enzymes.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered by addition, substitution, or deletion of a portion of a natural nucleic acid molecule or protein.

Reducing agent: An element or compound that loses “donates” an electron to another chemical species in a redox chemical reaction. A reducing agent is typically in one of its lower possible oxidation states, and is known as the electron donor. A reducing agent is oxidized, because it loses electrons in the redox reaction. Exemplary reducing agents include, but are not limited to, sodium dithionite, ascorbic acid, N-acetylcysteine, methylene blue, glutathione, cytochrome b5/b5-reductase, hydralazine, earth metals, formic acid and sulfite compounds.

Sequence identity/similarity: The identity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Silyl Ether: —OSiR^{a}R^{b}, wherein each of R^{a} and R^{b} independently is selected from hydrogen, aliphatic, heteroaliphatic, haloaliphatic, haloheteroaliphatic, aromatic, or an organic functional group.

Subject: Living multi-cellular organisms, including vertebrate organisms, a category that includes both human and non-human mammals.

Substantially free of 2,3-diphosphoglycerate: An isolated modified globin molecule (such as isolated, modified hemoglobin) that contains 2,3-diphosphoglycerate, if at all, only as a minor component or impurity. Generally, the term refers to containing less than 1% 2,3-diphosphoglycerate, such as less than 0.1%, less than 0.01%, or essentially 0% of 2,3-diphosphoglycerate.

Sulfinyl: —S(O)R^{a}, wherein R^{a} is selected from hydrogen, aliphatic, heteroaliphatic, haloaliphatic, haloheteroaliphatic, aromatic, or an organic functional group. In particular disclosed embodiments, the sulfinyl group can be sulfinic acid, having a structure —S(O)R^{a}, wherein R^{a} is a OH group; or a sulfinate, having a structure —S(O)R^{a}, wherein R^{a} is a OH group that has been deprotonated and the negative charge of the deprotonated oxygen atom may be balanced with an M⁺counter ion, wherein M⁺may be an alkali ion, such as K⁺, Na⁺, Li⁺; an ammonium ion, such as ³⁰N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. In yet some additional embodiments, the sulfinyl group can be sulfenic acid (—S(O)H) or the conjugate base thereof.

Sulfonyl: —SO₂R^{a}, wherein R^{a} is selected from hydrogen, aliphatic, heteroaliphatic, haloaliphatic, haloheteroaliphatic, aromatic, or an organic functional group. In particular disclosed embodiments, the sulfonyl group can be sulfonic acid, having a structure —S(O)₂R^{a}, wherein R^{a} is a OH group; or a sulfonate, having a structure —S(O)₂R^{a}, wherein R^{a} is a OH group that has been deprotonated and the negative charge of the deprotonated oxygen atom may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Sulfonamide: —SO₂NR^{a}R^{b} or —N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as +N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. Synthetic: Produced by artificial means in a laboratory, for example a synthetic polypeptide can be chemically synthesized in a laboratory.

Therapeutically acceptable salt: Salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid.

Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L- tartrate, trichloroacetate, trifluoroacetate, phosphate, glutamate, bicarbonate, para- toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Therapeutically effective amount: A quantity of compound or composition, for instance, a modified globin, sufficient to achieve a desired effect in a subject being treated. For instance, this can be the amount necessary to scavenge carbon monoxide in the blood or tissues, reduce the level of HbCO in the blood, and/or reduce one or more signs or symptoms associated with carbon monoxide poisoning. In some examples, the therapeutically effective amount is an amount necessary to reduce the level of HbCO in the blood by at least 10%, at least 20%, at least 30%, at least 40%, at least 50%, at least 60%, at least 70%, at least 80%, at least 90%, or at least 95%. The disclosed modified hemoglobin molecules are effective over a wide dosage range and, for example, dosages per day will normally fall within the range of from 0.001 to 2000 mg/kg, more usually in the range of from 0.01 to 1000 mg/kg. However, it will be understood that the effective amount administered will be determined by the physician in the light of the relevant circumstances including the condition to be treated, the choice of compound to be administered, and the chosen route of administration, and therefore the above dosage ranges are not intended to be limiting. A therapeutically effective amount of compound is typically an amount such that when it is administered in a physiologically tolerable excipient composition, it is sufficient to achieve an effective systemic concentration or local concentration in the tissue.

Thial: —C(S)H.

Thiocarboxylic acid: —C(O)SH, or —C(S)OH.

Thiocyanate: —S—CN or —N═C═S.

Thioester or Thionoester: —C(O)SR^{a} or —C(S)OR^{a} wherein R^{a} is selected from hydrogen, aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S-heteroaryl; or -aliphatic-S-aliphatic, -aliphatic-S-aromatic, -aromatic-S-aliphatic, or -aromatic-S-aromatic.

Thioketone: —C(S)R^{a} wherein R^{a} is selected from hydrogen, aliphatic, heteroaliphatic, haloaliphatic, haloheteroaliphatic, aromatic, or an organic functional group.

### III. Modified Globin Molecules and Compositions Thereof

Disclosed herein are modified globin molecules and compositions including such molecules. In some embodiments, disclosed are compositions that include a globin, such as myoglobin or hemoglobin, wherein at least about 85%, at least about 90%, at least about 95%, at least about 96%, at least about 97%, at least about 98%, or at least about 99% of the globin is in the relaxed state (R state). The modified globin molecule can be from any mammalian species, such as a human or veterinary species. The modified globin molecule, such as hemoglobin or myoglobin, can be human, bovine, canine, or porcine and can be isolated from the blood.

The biochemical properties of hemoglobin in the tense (T) and relaxed (R) states are provided below.

TABLE 1
Exemplary binding and dissociation constants for hemoglobin, cytochrome c oxidase and
myoglobin for CO, NO and O₂*

| | O₂ on | O₂ off | O2 K_{d} | CO on | CO off | CO K_{d} | NO on | NO off | NO K_{d} |
|---|---|---|---|---|---|---|---|---|---|
| Compound | (M⁻¹s⁻¹) | (s⁻¹⁾ | (M) | (M⁻¹s⁻¹) | (s⁻¹⁾ | (M) | (M⁻¹s⁻¹) | (s⁻¹⁾ | (M) |
| Hb R state | 5 × 10⁷ | 15 | 3 × 10⁻⁷ | 6 × 10⁶ | 0.012 | 1.7 × 10⁻⁹ | 2 × 10⁷ | 1.8 × 10⁻⁵ | 0.9 × 10⁻¹² |
| Hb T state | 4.5 × 10⁶ | 1900 | 4.2 × 10⁻⁴ | 8.3 × 10⁴ | 0.09 | 1.1 × 10⁻⁶ | 2 × 10⁷ | 3 × 10⁻³ | 1.5 × 10⁻¹⁰ |
| Cytochrome | 1 × 10⁸ | — | — | 7 × 10⁴ | 0.023 | 3 × 10⁻⁷ | 1 × 10⁸ | 0.13 | 1.3 × 10⁻⁹ |
| c oxidase | | | | | | | | | |
| Myoglobin | 1.4 × 10⁷ | 10 | 7.1 × 10⁻⁷ | 5 × 10⁵ | 0.17 | 3.4 × 10⁻⁷ | 1.7 × 10⁷ | 1.2 × 10⁻⁴ | 0.7 × 10¹¹ |

*From Cooper et al., Biochim Biophys Acta 1411(2-3): 290-309, 1999

In some embodiments, the composition includes a hemoglobin, wherein the hemoglobin is substantially free of 2,3-diphosphoglycerate (2,3-DPG). In some embodiments, the Hb has less than 1% 2,3-DPG, such as less than 0.1%, less than 0.01%, or essentially 0% 2,3-DPG. In some embodiments, the composition includes less than 0.1%, less than 0.01%, or essentially 0% 2,3-DPG.

The amino acid sequence of hemoglobin in vertebrates is highly conserved (Vitturi et al., Free Radic Biol Med 55:119-129, 2013, incorporated herein by reference). The β-93 cysteine (β93Cys) residue of hemoglobin has similar functions in, for example, human and canines (Acharya et al., Biochem J 405: 503-511, 2007, incorporated herein by reference). The amino acid sequence of the human alpha subunit is disclosed in GENBANK® Accession no. NP_0005049.1 (SEQ ID NO: 1), and the human beta subunit is disclosed in GENBANK® Accession No. CAG38767.1 (SEQ ID NO: 2). The DNA sequence is disclosed in GENBANK Accession No. DQ659148.1 (SEQ ID NO: 17). The amino acid sequences of hemoglobin alpha and beta subunits from humans and a variety of different species are provided below and set forth herein as SEQ ID NOs: 1-16. The mature forms of the alpha and beta subunits of hemoglobin lack the N-terminal methionine residue, which is removed following protein synthesis to produce the mature form. The numbering used herein for Lys40, Cys93, Asp94 and His146 refers to the position in the mature form of the proteins. For example, Lys40 is at position 41 in SEQ ID NO: 1 (the immature form of the human hemoglobin alpha subunit), but following processing, the lysine will be at position 40.

Human alpha subunit (SEQ ID NO: 1)
MVLSPADKTNVKAAWGKVGAHAGEYGAEALERMFLSFPTTKTYFPHFDLSHGSAQVKG
HGKKVADALTNAVAHVDDMPNALSALSDLHAHKLRVDPVNFKLLSHCLLVTLAAHLPAE
FTPAVHASLDKFLASVSTVLTSKYR
Human beta subunit (SEQ ID NO: 2)
MVHLTPEEKSAVTALWGKVNVDEVGGEALGRLLVVYPWTQRFFESFGDLSTPDAVMGNP
KVKAHGKKVLGAFSDGLAHLDNLKGTFATLSELHCDKLHVDPENI-RLLGNVLVCVLAHH
FGKEFTPPVQAAYQKVVAGVANALAHKYH
Canine alpha subunit (SEQ ID NO: 3):
VLSPADKTNIKSTWDKIGGHAGDYGGEALDRTFQSFPTTKTYFPHFDLSPGSAQVKAHGK
KVADALTTAVAHLDDLPGALSALSDLHAYKLRVDPVNFKLLSHCLLVTLACHHPTEFTPA
VHASLDKFFAAVSTVLTSKYR
Canine beta subunit (SEQ ID NO: 4):
VHLTAEEKSLVSGLWGKVNVDEVGGEALGRLLIVYPWTQRFFDSFGDLSTPDAVMSNAK
VKAHGKKVLNSFSDGLKNLDNLKGTFAKLSELHCDKLHVDPENFKLLGNVLVCVLAHHF
GKEFTPQVQAAYQKVVAGVANALAHKYH
Porcine alpha subunit (SEQ ID NO: 5):
VLSAADKANVKAAWGKVGGQAGAHGAEALERMFLGFPTTKTYFPHFNLSHGSDQVKAH
GQKVADALTKAVGHLDDLPGALSALSDLHAHKLRVDPVNFKLLSHCLLVTLAAHHPDDF
NPSVHASLDKFLANVSTVLTSKYR
Porcine beta subunit (SEQ ID NO: 6):
MVHLSAEEKEAVLGLWGKVNVDEVGGEALGRLLVVYPWTQRFFESFGDLSNADAVMGN
PKVKAHGKKVLQSFSDGLKHLDNLKGTFAKLSELHCDQLHVDPENFRLLGNVIVVVLARR
LGHDFNPNVQAAFQKVVAGVANALAHKYH
Equine alpha subunit (SEQ ID NO: 7):
MVLSAADKTNVKAAWSKVGGHAGEYGAEALERMFLGFPTTKTYFPHIDLSHGSAQVKA
HGKKVGDALTLAVGHLDDLPGALSNLSDLHAHKLRVDPVNFKLLSHCLLSTLAVHLPNDF
TPAVHASLDKFLSSVSTVLTSKYR
Equine beta subunit (SEQ ID NO: 8):
VQLSGEEKAAVLALWDKVNEEEVGGEALGRLLVVYPWTQRFFDSFGDLSNPGAVMGNP
KVKAHGKKVLHSFGEGVHHLDNLKGTFAALSELHCDKLHVDPENFRLLGNVLVVVLARH
FGKDFTPELQASYQKVVAGVANALAHKYH
Bovine alpha subunit (SEQ ID NO: 9):
MVLSAADKGNVKAAWGKVGGHAAEYGAEALERMFLSFPTTKTYFPHFDLSHGSAQVKG
HGAKVAAALTKAVEHLDDLPGALSELSDLHAHKLRVDPVNFKLLSHSLLVTLASHLPSDF
TPAVHASLDKFLANVSTVLTSKYR
Bovine beta subunit (SEQ ID NO: 10):
MLTAEEKAAVTAFWGKVKVDEVGGEALGRLLVVYPWTQRFFESFGDLSTADAVMNNPK
VKAHGKKVLDSFSNGMKHLDDLKGTFAALSELHCDKLHVDPENFKLLGNVLVVVLARNF
GKEFTPVLQADFQKVVAGVANALAHRYH
Murine alpha subunit (SEQ ID NO: 11):
MVLSGEDKSNIKAAWGKIGGHGAEYGAEALERMFASFPTTKTYFPHFDVSHGSAQVKGH
GKKVADALASAAGHLDDLPGALSALSDLHAHKLRVDPVNFKLLSHCLLVTLASHHPADFT
PAVHASLDKFLASVSTVLTSKYR
Murine beta subunit (SEQ ID NO: 12):
MVHLTDAEKAAVSCLWGKVNSDEVGGEALGRLLVVYPWTQRYFDSFGDLSSASAIMGN
AKVKAHGKKVITAFNDGLNHLDSLKGTFASLSELHCDKLHVDPENFRLLGNMIVIVLGHH
LGKDFTPAAQAAFQKVVAGVATALAHKYH
Feline alpha subunit (SEQ ID NO: 13):
VLSAADKSNVKACWGKIGSHAGEYGAEALERTFCSFPTTKTYFPHFDLSHGSAQVKAHGQ
KVADALTQAVAHMDDLPTAMSALSDLHAYKLRVDPVNFKFLSHCLLVTLACHHPAEFTP
AVHASLDKFFSAVSTVLTSKYR
Feline beta subunit (SEQ ID NO: 14):
GFLTAEEKGLVNGLWGKVNVDEVGGEALGRLLVVYPWTQRFFESFGDLSSADAIMSNAK
VKAHGKKVLNSFSDGLKNIDDLKGAFAKLSELHCDKLHVDPENI-RLLGNVLVCVLAHHF
GHDFNPQVQAAFQKVVAGVANALAHKYH
Rhesusmacaque alpha subunit (SEQ ID NO: 15):
MVLSPADKSNVKAAWGKVGGHAGEYGAEALERMFLSFPTTKTYFPHFDLSHGSAQVKGH
GKKVADALTLAVGHVDDMPNALSALSDLHAHKLRVDPVNFKLLSHCLLVTLAAHLPAEF
TPAVHASLDKFLASVSTVLTSKYR
Rhesusmacaque beta subunit (SEQ ID NO: 16):
VHLTPEEKNAVTTLWGKVNVDEVGGEALGRLLLVYPWTQRFFESFGDLSSPDAVMGNPK
VKAHGKKVLGAFSDGLNHLDNLKGTFAQLSELHCDKLHVDPENFKLLGNVLVCVLAHHF
GKEFTPQVQAAYQKVVAGVANALAHKYH

### Alignment of Alpha Subunits:

```
Porcine -VLSAADKANVKAAWGKVGGQAGAHGAEALERMFLGFPTTKTYFPHFNLSHGSDQVKAHG 59
Murine MVLSGEDKSNIKAAWGKIGGHGAEYGAEALERMFASFPTTKTYFPHFDVSHGSAQVKGHG 60
Bovine MVLSAADKGNVKAAWGKVGGHAAEYGAEALERMFLSFPTTKTYFPHFDLSHGSAQVKGHG 60
Equine MVLSAADKTNVKAAWSKVGGHAGEYGAEALERMFLGFPTTKTYFPHFDLSHGSAQVKAHG 60
Human MVLSPADKTNVKAAWGKVGAHAGEYGAEALERMFLSFPTTKTYFPHFDLSHGSAQVKGHG 60
Rhesus MVLSPADKSNVKAAWGKVGGHAGEYGAEALERMFLSFPTTKTYFPHFDLSHGSAQVKGHG 60
Canine -VLSPADKTNIKSTWDKIGGHAGDYGGEALDRTFQSFPTTKTYFPHFDLSPGSAQVKAHG 59
Feline -VLSAADKSNVKACWGKIGSHAGEYGAEALERTFCSFPTTKTYFPHFDLSHGSAQVKAHG 59
                    *** ** *:*: *.*:*.:.. :*.***:* * .***********::* ** ***.**
Porcine QKVADALTKAVGHLDDLPGALSALSDLHAHKLRVDPVNFKLLSHCLLVTLAAHHPDDFNP 119
Murine KKVADALASAAGHLDDLPGALSALSDLHAHKLRVDPVNFKLLSHCLLVTLASHHPADFTP 120
Bovine AKVAAALTKAVEHLDDLPGALSELSDLHAHKLRVDPVNFKLLSHSLLVTLASHLPSDFTP 120
Equine KKVGDALTLAVGHLDDLPGALSNLSDLHAHKLRVDPVNFKLLSHCLLSTLAVHLPNDFTP 120
Human KKVADALTNAVAHVDDMPNALSALSDLHAHKLRVDPVNFKLLSHCLLVTLAAHLPAEFTP 120
Rhesus KKVADALTLAVGHVDDMPNALSALSDLHAHKLRVDPVNFKLLSHCLLVTLAAHLPAEFTP 120
Canine KKVADALTTAVAHLDDLPGALSALSDLHAYKLRVDPVNFKLLSHCLLVTLACHHPTEFTP 119
Feline QKVADALTQAVAHMDDLPTAMSALSDLHAYKLRVDPVNFKFLSHCLLVTLACHHPAEFTP 119
                    **. **: *. *:**:* *:* ******:**********:***.** *** * * :*.*
Porcine SVHASLDKFLANVSTVLTSKYR 141 SEQ ID NO: 5
Murine AVHASLDKFLASVSTVLTSKYR 142 SEQ ID NO: 11
Bovine AVHASLDKFLANVSTVLTSKYR 142 SEQ ID NO: 9
Equine AVHASLDKFLSSVSTVLTSKYR 142 SEQ ID NO: 7
Human AVHASLDKFLASVSTVLTSKYR 142 SEQ ID NO: 1
Rhesus AVHASLDKFLASVSTVLTSKYR 142 SEQ ID NO: 15
Canine AVHASLDKFFAAVSTVLTSKYR 141 SEQ ID NO: 3
Feline AVHASLDKFFSAVSTVLTSKYR 141 SEQ ID NO: 13
                  :*******:: ***********
```

### Alignment of Beta Subunits:

```
Murine MVHLTDAEKAAVSCLWGKVNSDEVGGEALGRLLVVYPWTQRYFDSFGDLSSASAIMGNAK 60
Equine -VQLSGEEKAAVLALWDKVNEEEVGGEALGRLLVVYPWTQRFFDSFGDLSNPGAVMGNPK 59
Bovine --MLTAEEKAAVTAFWGKVKVDEVGGEALGRLLVVYPWTQRFFESFGDLSTADAVMNNPK 58
Porcine MVHLSAEEKEAVLGLWGKVNVDEVGGEALGRLLVVYPWTQRFFESFGDLSNADAVMGNPK 60
Feline -GFLTAEEKGLVNGLWGKVNVDEVGGEALGRLLVVYPWTQRFFESFGDLSSADAIMSNAK 59
Human MVHLTPEEKSAVTALWGKVNVDEVGGEALGRLLVVYPWTQRFFESFGDLSTPDAVMGNPK 60
Rhesus -VHLTPEEKNAVTTLWGKVNVDEVGGEALGRLLLVYPWTQRFFESFGDLSSPDAVMGNPK 59
Canine -VHLTAEEKSLVSGLWGKVNVDEVGGEALGRLLIVYPWTQRFFDSFGDLSTPDAVMSNAK 59
                        *: ** * :*.**: :***********:*******:*:******. .*:*.* *
Murine VKAHGKKVITAFNDGLNHLDSLKGTFASLSELHCDKLHVDPENFRLLGNMIVIVLGHHLG 120
Equine VKAHGKKVLHSFGEGVHHLDNLKGTFAALSELHCDKLHVDPENFRLLGNVLVVVLARHFG 119
Bovine VKAHGKKVLDSFSNGMKHLDDLKGTFAALSELHCDKLHVDPENFKLLGNVLVVVLARNFG 118
Porcine VKAHGKKVLQSFSDGLKHLDNLKGTFAKLSELHCDQLHVDPENFRLLGNVIVVVLARRLG 120
Feline VKAHGKKVLNSFSDGLKNIDDLKGAFAKLSELHCDKLHVDPENFRLLGNVLVCVLAHHFG 119
Human VKAHGKKVLGAFSDGLAHLDNLKGTFATLSELHCDKLHVDPENFRLLGNVLVCVLAHHFG 120
Rhesus VKAHGKKVLGAFSDGLNHLDNLKGTFAQLSELHCDKLHVDPENFKLLGNVLVCVLAHHFG 119
Canine VKAHGKKVLNSFSDGLKNLDNLKGTFAKLSELHCDKLHVDPENFKLLGNVLVCVLAHHFG 119
                  ********: :*.:*: ::*.***:** *******:********:****::* **.:.:*
Murine KDFTPAAQAAFQKVVAGVATALAHKYH 147 SEQ ID NO: 12
Equine KDFTPELQASYQKVVAGVANALAHKYH 146 SEQ ID NO: 8
Bovine KEFTPVLQADFQKVVAGVANALAHRYH 145 SEQ ID NO: 10
Porcine HDFNPNVQAAFQKVVAGVANALAHKYH 147 SEQ ID NO: 6
Feline HDFNPQVQAAFQKVVAGVANALAHKYH 146 SEQ ID NO: 14
Human KEFTPPVQAAYQKVVAGVANALAHKYH 147 SEQ ID NO: 2
Rhesus KEFTPQVQAAYQKVVAGVANALAHKYH 146 SEQ ID NO: 16
Canine KEFTPQVQAAYQKVVAGVANALAHKYH 146 SEQ ID NO: 4
                  ::*.* ** :********.****:**
```

It is disclosed herein that salt bridges between β-Asp94 and β-His146 and between β-His146 and α-Lys40 help generate the T state of hemoglobin (Hb). Covalent modification of the β-Cys93 residue of Hb, such as, but not limited to, with NEM, N-acetylcysteine, cysteine, glutathione, 3-mercapto-1,2,3-triazole, 2-mercapto-pyridyl, or similar molecules interrupts these salt bridges increasing affinity towards O₂ and CO by stabilizing the R state of Hb. Any of the methods disclosed below can be used to prepare a covalently modified Hb, which can be included in the present compositions.

In other embodiments, the modified hemoglobin is produced by reacting an isolated hemoglobin, such as hemoglobin isolated from mammalian blood or produced synthetically, with any suitable reactant as disclosed herein. Any suitable reaction conditions can be used to combine the Hb and the reactant, such as disulfide bond cleavage, alkylation (e.g., methylation or addition of other alkyl-containing groups), thiol-ene reactions (or alkene hydrothiolation, wherein a thiol group of the β-Cys93 residue is reacted with an alkene-containing compound in combination with a radical initiator or other catalyst, which is known to those of ordinary skill in the art as an embodiment of a “click” chemistry reaction), S-nitrosation (wherein a nitric oxide group is covalently attached to the thiol group of the β-Cys93 residue), or any combination thereof.

In some embodiments, the modified hemoglobin can be modified with reactants described herein so as to provide a modified hemoglobin having a structure as illustrated in FIG. 1 (see depiction of “R state Hb”). The reactant can become covalently bound to the Cys93 thiol moiety via carbon-sulfur bond formation or sulfur-sulfur bond formation; Cys93—S—R′, where:

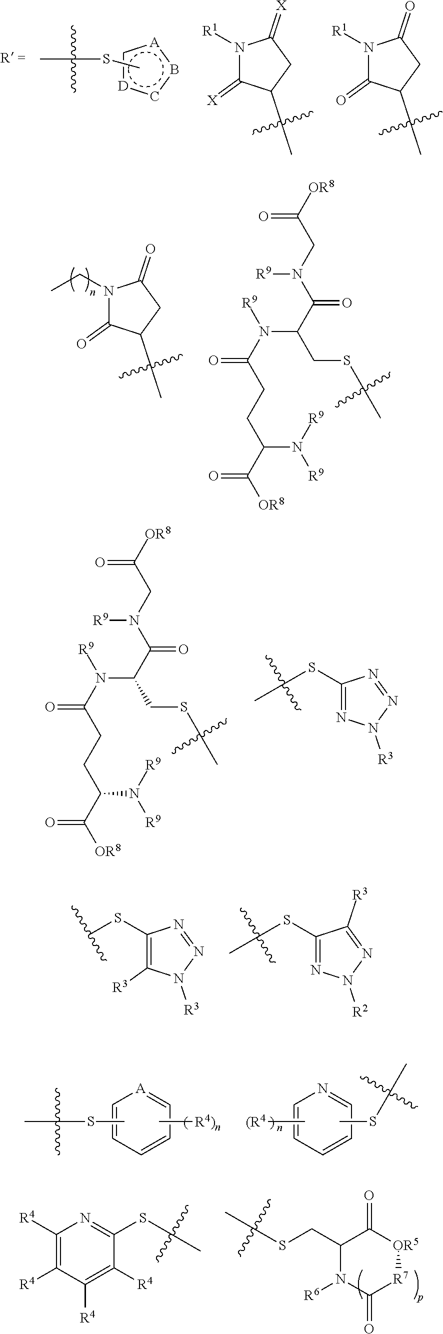

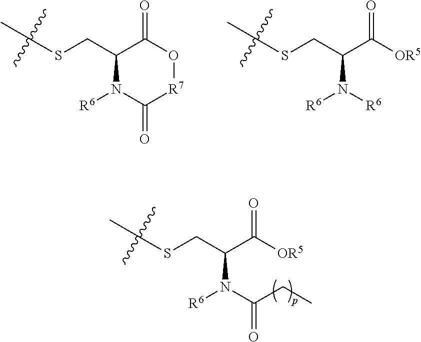

The modified hemoglobin can be a recombinantly derived hemoglobin resulting in a modification or removal of β-Cys93 or the β-Asp94 salt bridge partner, β-His146, or its other salt bridge partner, α-Lys40. In further embodiments, the modified hemoglobin includes modifications of β-Asp94, α-Lys40 (such as carbamylation or carbamoylation), and/or β-His146 residues to prevent salt bridges that would otherwise be interrupted by a modified β-Cys93. In such embodiments, the β-Cys93 need not participate in the salt bridges (that is, it need not be bound to or form any electrostatic interaction with the salt bridges) to facilitate interruption. In further embodiments, the α-Ala88 residue could be modified to a polar or protic amino acid such as Cys88 or Ser88 resulting in disruption of hydrogen bonding between α-Tyr140, β-Pro36, and β-Trp37 or a new hydrogen bond to α-His89, each destabilizes the T state.

Additionally, the addition of zinc to stripped hemoglobin or the addition of zinc to any of these modified hemoglobin molecules can serve to further increase their affinity for oxygen (Rifkind et al., Biochemistry 1977 Oct. 4; 16(20):4438-43).

In additional embodiments, disclosed are molecules that more effectively treat carbon monoxide poisoning than native hemoglobin. These specifically modified 2,3-DPG free hemoglobin preferentially bind CO from red blood cell encapsulated Hb and heme-containing proteins such as complex IV in mitochondria. The R form of hemoglobin allows for tighter bonding of CO and more efficient CO scavenging than the T state. Erythrocytic 2,3-DPG found in human red blood cells stabilizes the T-state of Hb. Stripped hemoglobin (StHb) that lacks 2,3-DPG, leading to R-state Hb, possesses increased affinity towards oxygen and CO. A disclosed hemoglobin, such as, but not limited to, a human, bovine, porcine, equine or canine hemoglobin that is modified at β-Cys93 can be used similarly. In some embodiments, the β-Cys93 is covalently modified with any one or more of the reactants disclosed herein. In some such embodiments, the hemoglobin can have a structure selected from

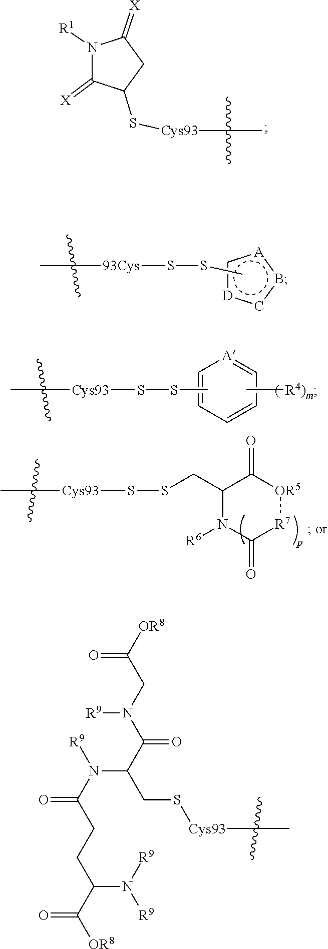

wherein

each X independently is selected from oxygen, sulfur, NR, or CRR′, wherein each R and R′ independently is hydrogen, aliphatic, heteroaliphatic, haloaliphatic, haloheteroaliphatic, aromatic, or a combination thereof;

R¹ is hydrogen, aliphatic, heteroaliphatic, haloaliphatic, haloheteroaliphatic, aromatic, or a combination thereof;

each of A, B, C, and D independently is C, CR³, N, NR², or O, wherein each of R² and R³ independently is hydrogen, aliphatic, heteroaliphatic, haloaliphatic, haloheteroaliphatic, aromatic, or a combination thereof;

A′ is N, CR⁴, or CH;

each R⁴ independently is aliphatic, heteroaliphatic, aromatic, an organic functional group, or any combination thereof;

m is an integer ranging from 0 to 5;

each of R⁵, R⁶, and R⁷ independently is hydrogen, aliphatic, heteroaliphatic, haloaliphatic, haloheteroaliphatic, aromatic, or a combination thereof;

the dotted line indicates an optional bond between the illustrated oxygen atom and the R⁷ group;

p can be 1 or 0 and when p is 0, the nitrogen atom is further bound to a second R⁶ group, which can be the same or different from the other R⁶ group;

each R⁸ independently is hydrogen, aliphatic, heteroaliphatic, haloaliphatic, haloheteroaliphatic, aromatic, or a combination thereof;

each R⁹ independently is hydrogen, aliphatic, heteroaliphatic, haloaliphatic, haloheteroaliphatic, aromatic, or a combination thereof; and

wherein the Cys93 is the β-Cys93 of the hemoglobin.

In particular disclosed embodiments, the β-Cys93 is covalently modified to have a structure selected from:

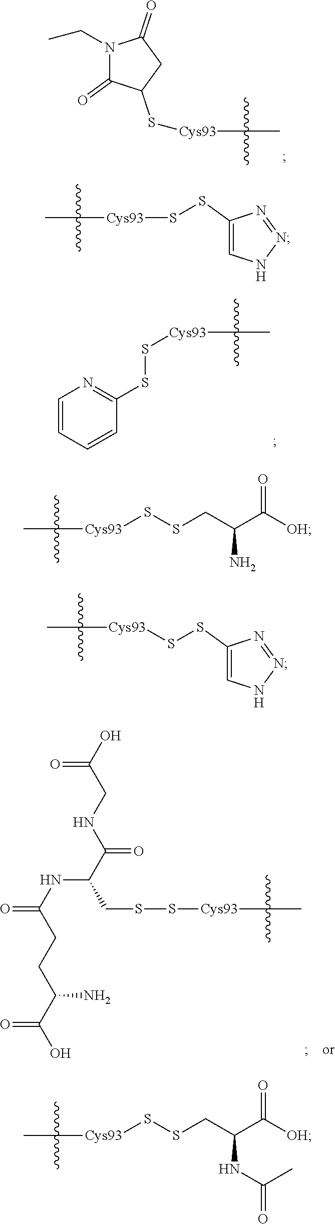

wherein the Cys93 is the β-Cys93 of the hemoglobin.

In some embodiments, the hemoglobin can include a terminal amino acid that comprises a functionalized amine moiety. In some embodiments, the functionalized amine moiety can be carbamylated (e.g., -CO₂ addition to amines), alkylated (e.g., methylation leading to alkylamine formation), protected via carbamation (such as functionalizing an amine group with a protecting group leading to a carbamate, wherein protecting groups can be, but are not limited to, tert-butoxycarbony (BOC) or fluorenylmethyloxycarbonyl (Fmoc)), carbamoylated (e.g., addition of a —C(O)NH₂ group), or combinations thereof.

One or more of the modified hemoglobins prepared using the methods below can be included in the compositions, without limitation.

In some embodiments, disclosed are pharmaceutical compositions that include one or more modified globins, such as modified hemoglobins, disclosed herein, or a derivative thereof, together with one or more pharmaceutically acceptable carriers thereof and optionally one or more other therapeutic ingredients. The excipient(s)/carrier(s) must be “acceptable” in the sense of being compatible with the other ingredients of the formulation and not deleterious to the recipient thereof.

Proper formulation of the pharmaceutical composition is dependent upon the route of administration chosen. Any of the well-known techniques and excipients may be used as suitable and as understood in the art. In some embodiments, the composition includes one or more of the following excipients: N-acetyl cysteine, sodium citrate, glycine, histidine, glutamic acid, sorbitol, maltose, mannitol, trehalose, lactose, glucose, raffinose, dextrose, dextran, ficoll, gelatin, hydroxyethyl starch, benzalkonium chloride, benzethonium chloride, benzyl alcohol, chlorobutanol, m-cresol, myristyl gamma-picolinium chloride, paraben methyl, paraben propyl, 2-penoxythanol, phenyl mercuric nitrate, thimerosal, acetone sodium bisulfite, argon, ascorbyl palmitate, ascorbate (sodium/acid), bisulfite sodium, butylated hydroxy anisole (BHA), butylated hydroxy toluene (BHT), cysteine/cysteinate HCl, dithionite sodium (Na hydrosulfite, Na sulfoxylate), gentisic acid, gentisic acid ethanolamine, glutamate monosodium, glutathione, formaldehyde sulfoxylate sodium, metabisulfite potassium, metabisulfite sodium, methionine, monothioglycerol (thioglycerol), nitrogen, propyl gallate, sulfite sodium, tocopherol alpha, alpha tocopherol hydrogen succinate, and thioglycolate sodium. The present disclosure also contemplates other excipients, including any disclosed in Pramanick et al., Pharma Times 45(3): 65-77, 2013, which is herein incorporated by reference.

The pharmaceutical compositions disclosed herein may be manufactured in any manner known in the art, e.g., by means of conventional mixing, dissolving, granulating, dragee-making, levigating, emulsifying, encapsulating, entrapping or compression processes. In some embodiments, the pharmaceutical compositions for use in accordance with embodiments herein can be formulated in a conventional manner using one or more physiologically acceptable carriers. The compositions can be prepared in a manner well known in the pharmaceutical arts, and can be administered by a variety of routes, depending upon whether local or systemic treatment is desired and upon the area to be treated.

The compositions include those suitable for parenteral (including subcutaneous, intradermal, intramuscular, intravenous, intraarticular, and intramedullary), or intraperitoneal administration although the most suitable route may depend upon for example the condition and disorder of the recipient. Parenteral administration includes intravenous, intraarterial, subcutaneous, intraperitoneal, intramuscular or injection or infusion; or intracranial, e.g., intrathecal or intraventricular, administration. Parenteral administration can be in the form of a single bolus dose, or may be, for example, by a continuous perfusion pump. In some embodiments, administration is intravenous.

Conventional pharmaceutical carriers, aqueous, thickeners and the like may be necessary or desirable. In some embodiments, the compounds can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, solubilizers, preservatives and the like. The artisan can refer to various pharmacologic references for guidance. For example, Modern Pharmaceutics, 5th Edition, Banker & Rhodes, CRC Press (2009); and Goodman & Gilman's The Pharmaceutical Basis of Therapeutics, 13th Edition, McGraw Hill, New York (2018) can be consulted. The compositions may conveniently be presented in unit dosage form and may be prepared by any of the methods well known in the art of pharmacy. Typically, these methods include the step of bringing into association a modified globin molecule disclosed herein, the carrier which constitutes one or more accessory ingredients. In general, the compositions are prepared by uniformly and intimately bringing into association the active ingredient with liquid carriers and then, if necessary, shaping the product into the desired composition.

The modified globin, such as modified hemoglobin, may be formulated for parenteral administration by injection. Compositions for injection may be presented in unit dosage form, e.g., in ampoules or in multi-dose containers, with an added preservative. The pharmaceutical compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described. Pharmaceutical compositions for parenteral administration include aqueous and non-aqueous (oily) sterile injection solutions of the active compounds which may contain antioxidants, buffers, bacteriostats and solutes which render the composition isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes.

Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Generally, an effective dose is included in a pharmaceutical composition.

It should be understood that in addition to the ingredients particularly mentioned above, the pharmaceutical compositions described above may include other agents conventional in the art having regard to the type of pharmaceutical composition.

In some embodiments, the pharmaceutical composition may comprise about 0.01% to about 50% of the modified globin, such as modified hemoglobin, disclosed herein. In some embodiments, the one or more modified globin is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition.

The isolated, modified globin, such as the modified hemoglobin, can be effective over a wide dosage range and can be generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

In some embodiments, the modified globin, such as modified hemoglobin, is in a therapeutically effective amount. In some embodiments, the therapeutically effective amount may be about 0.1 g to about 1000 g, about 0.1 g to about 900 g, about 0.1 g to about 800 g, about 0.1 g to about 700 g, about 0.1 g to about 600 g, about 0.1 g to about 500 g, about 0.1 g to about 400 g, about 0.1 g to about 300 g, about 0.1 g to about 200 g, about 0.1 g to about 100 g, 1 g to 100 g, 10 g to 100g, 50g to 100g, 50 g to 200g, or a range between any two of these values. In one specifically non-limiting example, 50-100 g is administered, such as to an adult human subject.

The amount of modified globin, such as modified hemoglobin, administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

In some embodiments, the pharmaceutical compositions administered to a subject can be in the form of pharmaceutical compositions described above. In some embodiments, these compositions can be sterilized by conventional sterilization techniques, or may be sterile filtered.

Aqueous solutions can be packaged for use as is, or lyophilized, the lyophilized preparation being combined with a sterile aqueous carrier prior to administration. In some embodiments, the pH of the isolated, modified globin molecule preparations is about 3 to about 11, about 5 to about 9, about 5.5 to about 6.5, or about 5.5 to about 7.5. It will be understood that use of certain of the foregoing excipients, carriers, or stabilizers will result in the formation of pharmaceutical salts.

Some embodiments herein are directed to a pharmaceutical composition comprising a modified globin molecule that is substantially free of 2,3-diphosphoglycerate, as disclosed herein, and a pharmaceutically acceptable excipient. Some embodiments herein are directed to a pharmaceutical composition comprising a modified hemoglobin that is substantially free of 2,3-diphosphoglycerate, as disclosed herein, and a pharmaceutically acceptable excipient. Some embodiments are directed to a pharmaceutical composition comprising a modified globin molecule that is substantially free of 2,3-diphosphoglycerate, a pharmaceutically acceptable carrier, and further comprising a reducing agent. In certain embodiments, the reducing agent is ascorbic acid, N-acetylcysteine, sodium dithionite, methylene blue, glutathione, B5/B5-reductase/NADH, or a combination thereof.

In certain embodiments, the pharmaceutical composition can be de-oxygenated by producing and maintaining the modified globin molecule, such as modified hemoglobin, or pharmaceutical composition in an oxygen free environment.

### IV. Modified Hemoglobin and Methods of Preparation

Disclosed herein are embodiments of a method of preparing an isolated, modified hemoglobin for therapeutic use. In some embodiments, the method includes obtaining whole blood, packed red blood cells, or a combination thereof and isolating hemoglobin molecules from the whole blood, packed red blood cells, or combination thereof. In some embodiments, the isolated hemoglobin molecules can be produced synthetically.

In some embodiments, the method comprises reacting the isolated hemoglobin with a reactant that is configured to form a chemical bond with the β-Cys93 residue of Hb hemoglobin to provide R-state Hb. In particular disclosed embodiments, the reactant is capable of forming a chemical bond with the β-Cys93 residue of Hb to thereby disrupt one or more salt bridges between β-Asp94 and β-His146 and/or between β-His146 and α-Lys40. In some embodiments, the reactant reacts with the β-Cys93 residue of Hb to provide a thioester group, a thioether group, a disulfide group, a sulfenate group, a sulfinate group, a sulfonate group, a sulfate group, or a nitrosothiol group (“—SNO”) between at least a portion of the reactant and the cysteine moiety of the Hb. In some embodiments, organometallic reactions resulting in thiometal bonding of the β-Cys93 residue can be used. Any suitable reaction conditions can be used to combine the Hb and the reactant, such as disulfide bond cleavage, alkylation (e.g., methylation or addition of other alkyl-containing groups), thiol-ene reactions (or alkene hydrothiolation, wherein a thiol group of the β-Cys93 residue is reacted with an alkene-containing compound in combination with a radical initiator or other catalyst, which is known to those of ordinary skill in the art as an embodiment of a “click” chemistry reaction), S-nitrosation (wherein a nitric oxide group is covalently attached to the thiol group of the β-Cys93 residue), or any combination thereof.

In some embodiments, the reactant is a chemical compound comprising at least one thiol group, at least one disulfide bond, or a sulfur-reactive functional group capable of forming a covalent bond with a sulfur atom of the β-Cys93 residue of Hb. In some embodiments, the sulfur-reactive functional group is a carbon-carbon double bond, a carbon-halide bond (e.g., —CR₂I, —CR₂Br, —CR₂F, —CR₂C₁, wherein each R independently is hydrogen, aliphatic, heteroaliphatic, haloaliphatic, haloheteroaliphatic, aromatic, or a combination thereof), a nitric oxide group, or other groups capable of providing a carbon-sulfur bond upon reaction with the β-Cys93 residue of Hb, such as methylating agents. In some embodiments, the sulfur-reactive functional group is a carbon-carbon double bond or a —CR₂I group (wherein each R independently is hydrogen, aliphatic, heteroaliphatic, haloaliphatic, haloheteroaliphatic, aromatic, or a combination thereof). In particular disclosed embodiments, the reactant is an iodoacetamide or a chemical compound having a structure satisfying any one or more of the below formulas. In some embodiments, the reactant is a chemical compound having a structure satisfying Formula I.

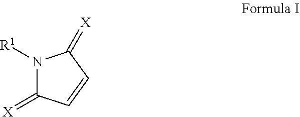

With reference to Formula I, each X independently can be selected from oxygen, sulfur, NR, or CRR′, wherein each R and R′ independently is hydrogen, aliphatic, heteroaliphatic, haloaliphatic, haloheteroaliphatic, aromatic, or a combination thereof; and R¹ is hydrogen, aliphatic, heteroaliphatic, haloaliphatic, haloheteroaliphatic, aromatic, or a combination thereof. In particular disclosed embodiments, each X independently is oxygen and R¹ is aliphatic, such as alkyl, alkenyl, or alkynyl.

In some embodiments, reactants having a structure satisfying Formula I also can have structures satisfying one or more of Formulas IA or IB, below.

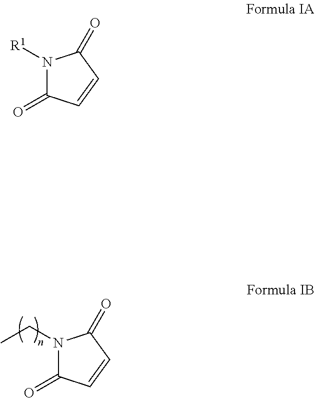

With reference to Formula IA, R¹ can be as recited above for Formula I. In some embodiments, R¹ is alkyl, such as methyl, ethyl, propyl, butyl, pentyl, hexyl, or the like. With reference to Formula IB, n can be an integer ranging from 1 to 20, such as 1 to 10, or 1 to 5, such as 1, 2, 3, 4, 5, 6, 7, 8, 9, or 10. In particular disclosed embodiments, n is 1. In exemplary embodiments, the reactant is N-ethylmaleimide.

In some embodiments, the reactant can have a structure satisfying Formula II below.

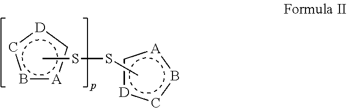

With reference to Formula II, each of A, B, C, and D independently can be C, CR³, N, NR², or O, wherein each of R² and R³ independently is hydrogen, aliphatic, heteroaliphatic, haloaliphatic, haloheteroaliphatic, aromatic, or a combination thereof; and p can be 1 or 0. When p is 0, the remaining sulfur atom is further bound to a hydrogen atom. In particular disclosed embodiments, at least two of A, B, C, and D are N, one of A, B, C, and D is CR³, and one of A, B, C, and D is NR², wherein each of R² and R³ independently is hydrogen or aliphatic (e.g., such as alkyl, alkenyl, or alkynyl). In some embodiments, each of A, B, C, and D can be selected so as to provide a diazole, a triazole, a tetrazole, an oxazole, an isoxazole, or other five-membered heteraromatic group.

In some embodiments, reactants having a structure satisfying Formula II also can have structures satisfying one or more of Formulas IIA-IID, below.

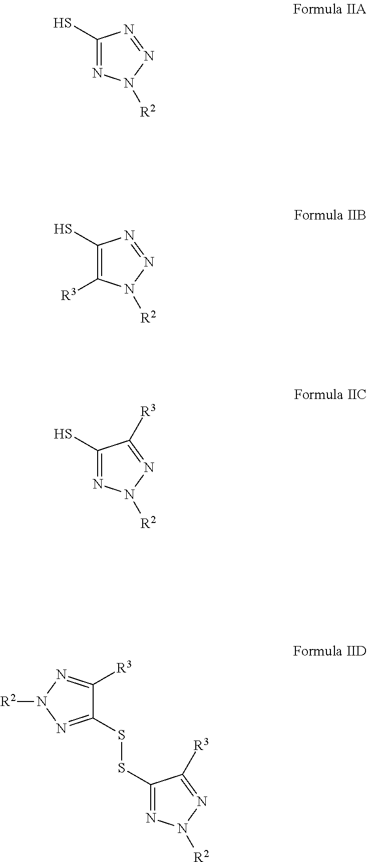

With reference to Formula IIA-IID, each of R² and R³ independently can be hydrogen, aliphatic, heteroaliphatic, haloaliphatic, haloheteroaliphatic, aromatic, or a combination thereof. In particular disclosed embodiments, each R² and R³ independently is hydrogen. In exemplary embodiments, the reactant is 4-4′-di(1,2,3-triazole) disulfide hydrate or 3-mercapto-1,2,3-triazole.

In yet additional embodiments, the reactant can have a structure satisfying Formula III below.

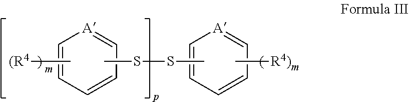

With reference to Formula III, each A′ independently can be N, CR⁴, or CH; each R⁴ independently can be aliphatic, heteroaliphatic, aromatic, an organic functional group, or any combination thereof; m can be an integer ranging from 0 to 5, such as 0 to 4, or 0 to 3, or 0 to 2, such as 0, 1, 2, 3, 4, or 5; and p can be 1 or 0. When p is 0, the remaining sulfur atom is further bound to a hydrogen atom.

In some embodiments, reactants having a structure satisfying Formula III also can have structures satisfying one or more of Formulas IIIA-IIID, below.

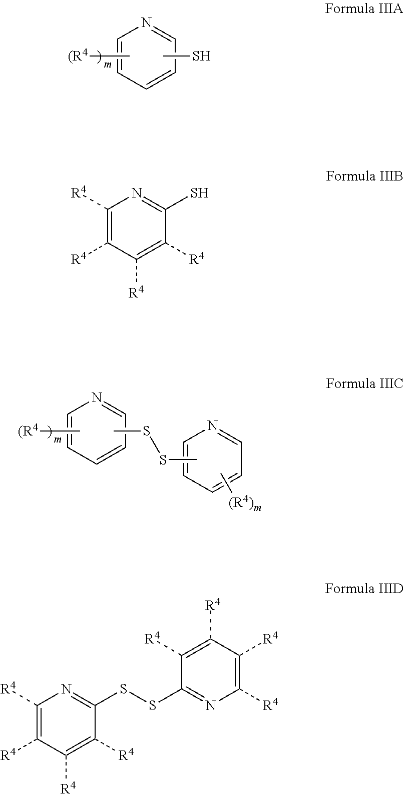

With reference to Formulas IIIA-IIID, each R⁴ independently can be selected from aliphatic, heteroaliphatic, aromatic, an organic functional group, or any combination thereof. In Formulas IIIB and IIID, the dotted lines represent optional bonds such that R⁴ can be present, and bound to the illustrated carbon atom, or R⁴ is not present and a hydrogen atom is bound to the corresponding atom. In particular disclosed embodiments of Formulas IIIA and IIIC, m is 0; and in particular disclosed embodiments of Formulas IIIB and IIID, no R⁴ groups are present. In exemplary embodiments, the reactant is 2,2′-dithiopyridine or 2-mercapto-pyridyl.

In yet additional embodiments, the reactant can have a structure satisfying Formula IV below.

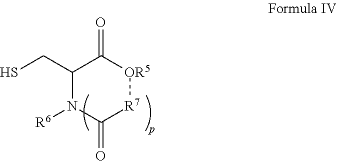

With reference to Formula IV, each of R⁵, R⁶, and R⁷ independently can be hydrogen, aliphatic, heteroaliphatic, haloaliphatic, haloheteroaliphatic, aromatic, or a combination thereof; p can be 1 or 0; and the dotted line indicates an optional bond between the illustrated oxygen atom and the R⁷ group. When p is 0, the illustrated nitrogen atom is further bound to a second R⁶ group, which can be the same or different from the other R⁶ group. Both enantiomers are contemplated.

In some embodiments, reactants having a structure satisfying Formula IV also can have structures satisfying one or more of Formulas IVA-IVC, below.

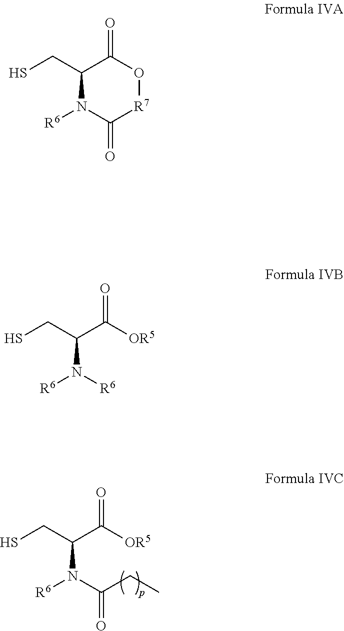

With reference to Formula IVA, R⁶ and R⁷ can be as described above for Formula IV. In some embodiments, R⁶ is hydrogen or aliphatic (e.g., alkyl, such as methyl, ethyl, propyl, or butyl); and R⁷ is CH₂. With reference to Formula IVB, R⁵ and R⁶ can be as described above for Formula IV.

In particular embodiments of Formula IVB, each of R⁵ and R⁶ is hydrogen. With reference to Formula IVC, R⁵ and R⁶ can be as described above for Formula IV and n can be an integer ranging from 0 to 20, such as 0 to 10, or 1 to 5, such as 0, 1, 2, 3, 4, 5, 6, 7, 8, 9, or 10. In particular disclosed embodiments of Formula IVC, R⁶ is hydrogen or aliphatic (e.g., alkyl, such as methyl, ethyl, propyl, or butyl); R⁵ is hydrogen; and n is 0. While one particular enantiomer is illustrated (the L-enantiomer) for the formulas above, the other enantiomer (the D-enantiomer) also is contemplated by the present disclosure. In exemplary embodiments, the reactant is acetylcysteine or cysteine.

In yet additional embodiments, the reactant can have a structure satisfying Formula V below.

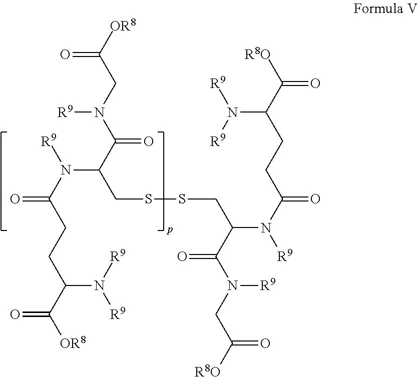

With reference to Formula V, each R⁸ independently can be hydrogen, aliphatic, heteroaliphatic, haloaliphatic, haloheteroaliphatic, aromatic, or a combination thereof; each R⁹ independently can be hydrogen, aliphatic, heteroaliphatic, haloaliphatic, haloheteroaliphatic, aromatic, or a combination thereof; and p can be 1 or 0. When p is 0, the remaining sulfur atom is further bound to a hydrogen atom. All possible stereoisomers are contemplated.

In some embodiments, reactants having a structure satisfying Formula V also can have structures satisfying one or more of Formulas VA and VB, below.

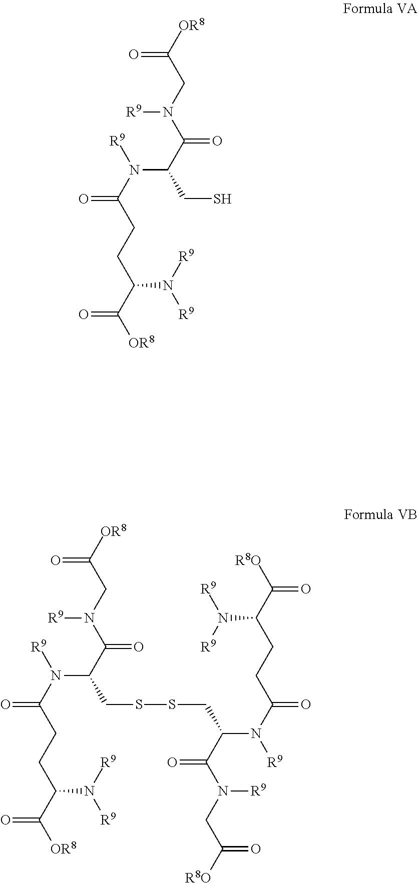

With reference to Formulas VA and VB, each R⁸ and R⁹ independently can be as recited above for Formula V. In particular embodiments, each R⁸ independently is hydrogen or aliphatic (e.g., alkyl, such as methyl, ethyl, propyl, or butyl); and each R⁹ independently is hydrogen or aliphatic (e.g., alkyl, such as methyl, ethyl, propyl, or butyl). In particular embodiments all R⁸ and R⁹ groups are hydrogen. While a particular stereoisomer is illustrated, all other possible stereoisomers are contemplated. In exemplary embodiments, the reactant is glutathione or diglutathione.

Any one or more of the above reactants can be reacted with Hb to form a covalent bond between the Hb and the reactant. As such, the Hb becomes covalently bound with the reactant to provide a covalently modified Hb. Compositions are disclosed herein that includes these covalently modified Hb.

Some embodiments further comprise filtering the treated hemoglobin to remove excess reactant and form a filtered hemoglobin. Some embodiments further comprise reacting the filtered hemoglobin with a reducing agent to form a modified hemoglobin. Some embodiments further comprise the removal of the reducing agent through column filtration or other means, versus retention of reducing agent in solution. Some embodiments further comprise placing the modified hemoglobin in an oxygen-free environment.

Disclosed herein is a method of preparing a modified hemoglobin for therapeutic use. In some embodiments, the method comprises, obtaining whole blood, packed red blood cells, or a combination thereof and isolating hemoglobin molecules from the whole blood, packed red blood cells, or a combination thereof. In some embodiments, the hemoglobin molecules can be produced synthetically.

In some embodiments, the method includes reacting the hemoglobin with a reactant selected from 2,2′-dithiopyridine/4-4′-di(1,2,3-triazole) disulfide hydrate, N-ethylmaleimide, N-acetylcysteine, cysteine, glutathione, 3-mercapto-1,2,3-triazole, 2-mercapto-pyridyl, a similar reactant or any combination thereof, to break disulfide bridges and form treated hemoglobin which is covalently modified. Some embodiments further comprise filtering the treated isolated hemoglobin to remove excess reactant and form a filtered isolated hemoglobin. Some embodiments further comprise reacting the filtered isolated hemoglobin with a reducing agent to form an isolated modified hemoglobin. Some embodiments further comprise the removal of the reducing agent through column filtration or other means, versus retention of reducing agent in solution. Some embodiments further comprise placing the isolated modified hemoglobin in an oxygen-free environment.

In some embodiments of the method of preparing a modified hemoglobin for therapeutic use, the whole blood, packed red blood cells, or a combination thereof are of human, bovine, equine, or porcine origin.

In some embodiments, naturally occurring hemoglobin is isolated from whole blood or packed red blood cells, (from human, bovine, equine, or porcine sources) by breaking apart the cells, and separating out and isolating the hemoglobin molecules. This process removes 2,3-DPG from the hemoglobin solution. The hemoglobin molecule is treated with 2,2′-dithiodipyridine (2-DPS, 220.31 g/mol) creating 2-mercaptopyridyl Hb (2MP-Hb). 2MP-Hb is gel filtered with a G25 column to remove excess 2-DPS and diluted with PBS. The 2MP-Hb is then reacted with excess thiol modifying agent dissolved in PBS. The modified Hb molecule is then concentrated. Alternatively, for triazole modifications, 4,4′-di(1,2,3-triazole) disulfide hydrate (4-DTD), MW: ˜236 g/mol for dihydrate) in the same manner as 2-DPS, yielding 4-triazoyl Hb (4-MTri-Hb). This is then combined with a triazole solution. Alternatively, NEM molecules can be reacted directly to the stripped Hb molecule to create NEM-Hb. The molecule will need to be reduced and maintained in reduced form. This can be achieved through adding a reducing agent with or without removal through a process such as a G25 gel separation column This reduced molecule can then be maintained in the reduced form with or without an additional reducing agent. This molecule can also be reduced through mechanical, electronic or photoactive method.

### V. Methods of Treating Carboxyhemoglobinemia

Methods are provided for treating carboxyhemoglobinemia in a subject. The methods include selecting a subject with carboxyhemoglobinemia and administering to the subject a therapeutically effective amount of a composition including a modified globin, such as a modified hemoglobin as disclosed herein, in its reduced form.

Also provided herein are methods of removing carbon monoxide from hemoglobin in blood or animal tissue. The methods include contacting the subject's blood or tissue with a modified globin molecule, such as a modified hemoglobin, as disclosed herein, or a pharmaceutical composition including the modified globin, such as the modified hemoglobin, as disclosed herein, in its reduced form.

In some embodiments, the method is an in vivo method, where contacting the blood or animal tissue with a modified globin molecule, such as a modified hemoglobin, includes administering a therapeutically effective amount of a composition including the modified globin molecule, such as the modified hemoglobin, to a subject. In some examples, the method further includes selecting a subject with carboxyhemoglobinemia prior to administering the composition comprising the modified globin molecule, such as the modified hemoglobin, to the subject. In some examples, the selected subject with carboxyhemoglobinemia has at least 5%, at least 10%, at least 15%, at least 20%, at least 30%, at least 40% or at least 50% carboxyhemoglobin in their blood. In particular non-limiting examples, the globin protein is a human globin protein, such as human hemoglobin, human myoglobin, human neuroglobin or human cytoglobin. In other non-limiting examples, the globin protein is from a non-human animal, such as a bovine globin protein or an equine globin protein.

In other embodiments, the method of removing carbon monoxide from hemoglobin in blood or animal tissue is an in vitro method.

In some embodiments, a composition is utilized that includes a globin, such as myoglobin or hemoglobin, wherein at least about 85%, 90%, 95%, 96%, 97%, 98%, or 99% of the globin is in the relaxed state. In some embodiments, the composition includes a hemoglobin, wherein the hemoglobin is substantially free of 2,3-DPG. In some embodiments, this includes less than 1% 2,3-DOG, such as less than 0.1%, less than 0.01%, or essentially 0% of 2,3-DPG. The composition that is utilized can include any modified globin disclosed herein, such as a modified hemoglobin as disclosed herein. The modified globin, such as hemoglobin, can be from any mammalian species, such as human and veterinary species. The modified globin molecule, such as hemoglobin or myoglobin, can be human, bovine, canine, equine, or porcine.

It is not necessary for 100% of the modified globin included in the composition to be reduced in order for the modified globin to be considered in reduced form. In some embodiments, at least 70% of the modified globin in the composition is reduced, such as at least 75%, at least 80%, at least 85%, at least 90%, at least 95%, at least 96%, at least 97%, at least 98% or at least 99%. In particular embodiments, 75-100%, 80-100%, 85-100%, 90-100% or 95-100% of the modified globin in the composition is reduced.

In some embodiments, the composition further includes a reducing agent. The reducing agent can be any reducing agent that can be safely administered to a subject, such as a human subject (for example, an agent with minimal and/or tolerable toxicity). In some examples, the reducing agent includes sodium dithionite, ascorbic acid, N-acetylcysteine, methylene blue, glutathione, cytochrome b5/b5-reductase, hydralazine, or any combination thereof. In some embodiments, the method further includes adding a second reducing agent to the composition. In most cases, the second reducing agent is added to the composition at a concentration that is the lowest effective concentration (for maintaining the modified globin in its reduced form) that is safely tolerated physiologically, such as by a human. In some examples, the concentration of reducing agent in the composition is about 10 μM to about 100 mM, such as about 50 μM to about 50 mM, about 100 μM to about 25 mM, about 250 μM to about 10 mM, about 500 μM to about 5 mM or about 750 μM to about to about 1 mM. In particular examples, the concentration of the reducing agent in the composition is no more than about 1.0 mM, no more than about 1.5 mM, no more than about 2.0 mM or no more than about 2.5 mM.

In some embodiments, the modified globin is hemoglobin. In other examples, the modified globin is myoglobin. In yet other examples, the modified globin is neuroglobin or cytoglobin. In particular non-limiting examples, the globin protein is a human globin protein, such as human hemoglobin, human myoglobin, human neuroglobin or human cytoglobin. In other non-limiting examples, the globin is from a non-human animal, such as a bovine globin protein or an equine globin protein.

In some embodiments of the method for removing carbon monoxide from hemoglobin in blood or animal tissue, the composition further includes a reducing agent. The reducing agent can be any reducing agent that can be safely administered to a subject, such as a human subject (for example, an agent with minimal and/or tolerable toxicity). In some examples, the reducing agent includes sodium dithionite, ascorbic acid, N-acetylcysteine, methylene blue, glutathione, cytochrome b5/b5-reductase, hydralazine, or any combination thereof.

In specific non-limiting examples, the modified globin molecule, as disclosed herein, or a pharmaceutical composition containing an isolated, modified globin molecule, as disclosed herein, is administered at a dose of from 0.1g to 300 g per day.

### VI. Methods of Treating Cyanide Poisoning

Cyanide is known to inhibit mitochondrial respiration, in a similar manner to CO-mediated inhibition of mitochondrial respiration by binding to the heme a3 center in cytochrome c oxidase. Although it partially binds the reduced form, cyanide binds strongest to the oxidized state of cytochrome c oxidase (complex IV of the electron transport chain) (Leavesley et al., Toxicol Sci 101(1):101-111, 2008). Similar to the ability of oxygen carriers to scavenge CO in the reduced state, oxygen carriers in the oxidized state, mediated through an oxidizing agent, are able to scavenge cyanide. Thus, the use of natural and artificial oxygen carriers for removing cyanide from cyano-hemoglobin located inside red blood cells, as well as other heme containing proteins in the body (such as cytochrome c oxidase), is contemplated herein.

Provided herein are methods of treating cyanide poisoning in a subject. In some embodiments, the method includes selecting a subject with cyanide poisoning; and administering to the subject the disclosed modified globin, such as modified hemoglobin, in its oxidized form.

Also provided herein are methods of removing cyanide from a heme-containing protein in blood or animal tissue. The methods include contacting the blood or animal tissue with a composition that includes a modified globin in its oxidized form. In some embodiments, the heme-containing protein is hemoglobin or cytochrome c oxidase.

In some embodiments, the method is an in vivo method, where contacting the blood or animal tissue with a composition comprising a modified globin includes administering a therapeutically effective amount of the composition to a subject. In some examples, the method further includes selecting a subject with cyanide poisoning prior to administering the composition to the subject.

In other embodiments, the method of removing cyanide from a heme-containing protein in blood or animal tissue is an in vitro method.

In some embodiments, a composition is utilized that includes a globin, such as myoglobin or hemoglobin, wherein at least about 85%, 90%, 95%, 96%, 97%, 98%, or 99% of the globin is in the relaxed state. In some embodiments, the composition includes a hemoglobin, wherein the hemoglobin is substantially free of 2,3-diphosphoglycerate. In some embodiments, this includes less than 1% 2,3-diphosphoglycerate, such as less than 0.1%, less than 0.01%, or essentially 0% of 2,3-diphosphoglycerate. The composition can include any modified globin disclosed herein, such as a modified hemoglobin as disclosed herein. The modified globin, such as hemoglobin, can be from any mammalian species, such as human and veterinary species. The modified globin molecule, such as hemoglobin or myoglobin, can be human, bovine, canine, equine, or porcine.

It is not necessary for 100% of the modified globin in the composition to be oxidized to be considered in oxidized form. In some embodiments, at least 70% of the modified globin is oxidized, such as at least 75%, at least 80%, at least 85%, at least 90%, at least 95%, at least 96%, at least 97%, at least 98% or at least 99%. In particular embodiments, 75-100%, 80-100%, 85-100%, 90-100% or 95-100% of the modified globin in the composition is oxidized.

In some embodiments, a composition is used that further includes an oxidizing agent. The oxidizing agent can be any oxidizing agent that can be safely administered to a subject, such as a human subject (for example, an agent with minimal and/or tolerable toxicity). In some examples, the oxidizing agent includes an oxygen-containing gas mixture, an oxygen-containing liquid mixture, a ferricyanide salt (such as potassium ferricyanide), or any combination thereof. In some embodiments, the method further includes adding a second oxidizing agent to the composition. In most cases, the second oxidizing agent is added to the composition at a concentration that is the lowest effective concentration (for maintaining the modified globin in its oxidized form) that is safely tolerated physiologically, such as by a human. In some examples, the concentration of oxidizing agent in the composition is about 10 μM to about 100 mM, such as about 50 μM to about 50 mM, about 100 μM to about 25 mM, about 250 μM to about 10 mM, about 500 μM to about 5 mM or about 750 μM to about to about 1 mM. In particular examples, the concentration of the oxidizing agent in the composition is no more than about 1.0 mM, no more than about 1.5 mM, no more than about 2.0 mM or no more than about 2.5 mM.

In some embodiments of the method for removing cyanide from a heme-containing protein in blood or animal tissue, the composition further includes an oxidizing agent. The oxidizing agent can be any oxidizing agent that can be safely administered to a subject, such as a human subject (for example, an agent with minimal and/or tolerable toxicity). In some examples, the oxidizing agent includes an oxygen-containing gas mixture, an oxygen-containing liquid mixture, a ferricyanide salt (such as potassium ferricyanide), or any combination thereof.

In some embodiments of the method for removing cyanide from a heme-containing protein in blood or animal tissue, the composition includes a modified globin protein as disclosed herein.

In some examples, the modified globin protein is a modified hemoglobin. In other examples, the modified globin protein is a modified myoglobin. In other non-limiting examples, the globin protein is from a non-human animal, such as a bovine globin protein or an equine globin protein.

In specific non-limiting examples, the modified globin molecule, as disclosed herein, or a pharmaceutical composition containing an isolated, modified globin molecule, as disclosed herein, is administered at a dose of from 0.1g to 300 g per day.

### VII. Methods of Treating Hydrogen Sulfide (H₂S) Poisoning

Hydrogen sulfide is known to inhibit mitochondrial respiration, in a similar manner to CO-mediated inhibition of mitochondrial respiration. H₂S binds strongest to the reduced form of cytochrome c oxidase (complex IV of the electron transport chain) (Nicholls et al., Biochem Soc Trans 41(5):1312-1316, 2013). Similar to an oxygen carrier's ability to scavenge CO in the reduced state, oxygen carriers in the reduced state, mediated through a reducing agent, are able to scavenge H₂S. Thus, the use of the disclosed compositions for removing H₂Sfrom hemoglobin located inside red blood cells, as well as other heme containing proteins in the body (such as cytochrome c oxidase), is contemplated herein.

Provided herein are methods of treating hydrogen sulfide (H₂S) poisoning in a subject. In some embodiments, the method includes selecting a subject with H₂S poisoning; and administering to the subject a therapeutically effective amount of a composition comprising a modified globin protein, as disclosed herein, in its reduced form.

Also provided herein are methods of removing H₂S from a heme-containing protein in blood or animal tissue. The methods include contacting the blood or animal tissue with a composition as disclosed herein. In some embodiments, the composition includes a modified globin protein, such as a modified hemoglobin or myoglobin.

In some embodiments, the method is an in vivo method, where contacting the blood or animal tissue with a composition comprising a modified globin includes administering a therapeutically effective amount of the composition to a subject. In some examples, the method further includes selecting a subject with H₂Spoisoning prior to administering the composition to the subject.

In other embodiments, the method of removing H₂Sfrom a heme-containing protein in blood or animal tissue is an in vitro method.

In some embodiments, a composition is utilized that includes a modified globin, such as a modified myoglobin or hemoglobin, wherein at least about 85%, 90%, 95%, 96%, 97%, 98%, or 99% of the globin in the composition is in the relaxed state. In some embodiments, the composition includes a hemoglobin, wherein the hemoglobin is substantially free of 2,3-diphosphoglycerate. In some embodiments, this includes less than 1% 2,3-diphosphoglycerate, such as less than 0.1%, less than 0.01%, or essentially 0% of 2,3-diphosphoglycerate. The composition that is utilized can include any modified globin disclosed herein, such as a modified hemoglobin as disclosed herein. The modified globin, such as hemoglobin, can be from any mammalian species, such as human and veterinary species. The modified globin molecule, such as hemoglobin or myoglobin, can be human, bovine, canine, equine, or porcine.

It is not necessary for 100% of the modified globin included in the composition to be reduced in order for the modified globin to be considered in reduced form. In some embodiments, at least 70% of the modified globin in the composition is reduced, such as at least 75%, at least 80%, at least 85%, at least 90%, at least 95%, at least 96%, at least 97%, at least 98% or at least 99%. In particular embodiments, 75-100%, 80-100%, 85-100%, 90-100% or 95-100% of the modified globin in the composition is reduced.

In some embodiments, the composition further includes a reducing agent. The reducing agent can be any reducing agent that can be safely administered to a subject, such as a human subject (for example, an agent with minimal and/or tolerable toxicity). In some examples, the reducing agent includes sodium dithionite, ascorbic acid, N-acetylcysteine, methylene blue, glutathione, cytochrome b5/b5-reductase, hydralazine, or any combination thereof. In some embodiments, the method further includes adding a second reducing agent to the composition. In most cases, the second reducing agent is added to the composition at a concentration that is the lowest effective concentration (for maintaining the modified globin in its reduced form) that is safely tolerated physiologically, such as by a human. In some examples, the concentration of reducing agent in the composition is about 10 μM to about 100 mM, such as about 50 μM to about 50 mM, about 100 μM to about 25 mM, about 250 μM to about 10 mM, about 500 μM to about 5 mM or about 750 μM to about to about 1 mM. In particular examples, the concentration of the reducing agent in the composition is no more than about 1.0 mM, no more than about 1.5 mM, no more than about 2.0 mM or no more than about 2.5 mM.

In some embodiments, the composition that is administered includes a modified globin protein. In some examples, the modified globin protein is a modified hemoglobin or myoglobin as disclosed herein. In yet other examples, the globin protein includes neuroglobin or cytoglobin. In particular non-limiting examples, the modified globin protein is a human globin protein, such as human hemoglobin, human myoglobin, human neuroglobin or human cytoglobin. In other non-limiting examples, the globin protein is from a non-human animal, such as a canine, porcine, bovine, or equine modified globin.

In specific non-limiting examples, the modified globin molecule, as disclosed herein, or a pharmaceutical composition containing an isolated, modified globin molecule, as disclosed herein, is administered at a dose of from 0.1g to 300 g per day.

### VIII. Specific Embodiments

Embodiment 1. A composition comprising a globin in a relaxed state, wherein at least 85% of the globin is in the relaxed state.

Embodiment 2. The composition of Embodiment 1, wherein the globin is myoglobin or hemoglobin.

Embodiment 3. The composition of Embodiment 1 or Embodiment 2, wherein the globin is hemoglobin.

Embodiment 4. The composition of Embodiment 3, wherein the hemoglobin is substantially free of 2,3-diphosphoglycerate.

Embodiment 5. The composition of any one of Embodiments 2-4, wherein the hemoglobin comprises a β-Cys93 that is covalently modified to inhibit one or both salt bridges between β-Asp94, β-His146 and α-Lys40.

Embodiment 6. The composition of Embodiment 5, wherein the β-Cys93 is covalently modified to have a structure satisfying any one or more of the following formulas:

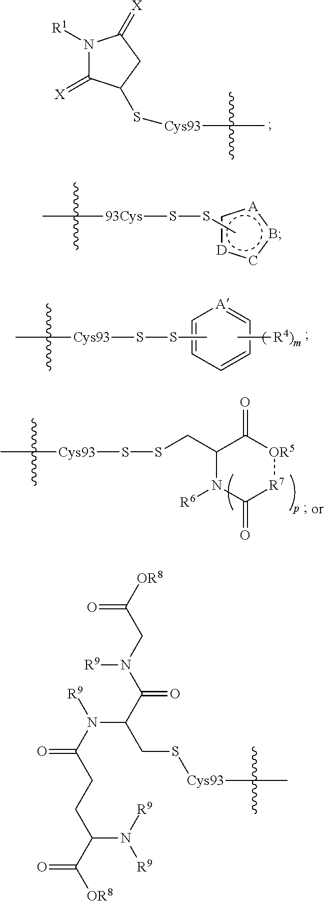

wherein

each X independently is selected from oxygen, sulfur, NR, or CRR′, wherein each R and R′ independently is hydrogen, aliphatic, heteroaliphatic, haloaliphatic, haloheteroaliphatic, aromatic, or a combination thereof;

R¹ is hydrogen, aliphatic, heteroaliphatic, haloaliphatic, haloheteroaliphatic, aromatic, or a combination thereof;

each of A, B, C, and D independently is C, CR³, N, NR², or 0, wherein each of R² and R³ independently is hydrogen, aliphatic, heteroaliphatic, haloaliphatic, haloheteroaliphatic, aromatic, or a combination thereof;

A′ is N, CR⁴, or CH;

each R⁴ independently is aliphatic, heteroaliphatic, aromatic, an organic functional group, or any combination thereof;

m is an integer ranging from 0 to 5;

each of R⁵, R⁶, and R⁷ independently is hydrogen, aliphatic, heteroaliphatic, haloaliphatic, haloheteroaliphatic, aromatic, or a combination thereof;

the dotted line indicates an optional bond between the illustrated oxygen atom and the R⁷ group;

p can be 1 or 0 and when p is 0, the nitrogen atom is further bound to a second R⁶ group, which can be the same or different from the other R⁶ group;

each R⁸ independently is hydrogen, aliphatic, heteroaliphatic, haloaliphatic, haloheteroaliphatic, aromatic, or a combination thereof;

each R⁹ independently is hydrogen, aliphatic, heteroaliphatic, haloaliphatic, haloheteroaliphatic, aromatic, or a combination thereof; and

wherein the Cys93 is the β-Cys93 of the hemoglobin.

Embodiment 7. The composition of Embodiment 5 or Embodiment 6, wherein the β-Cys93 is covalently modified to have a structure selected from:

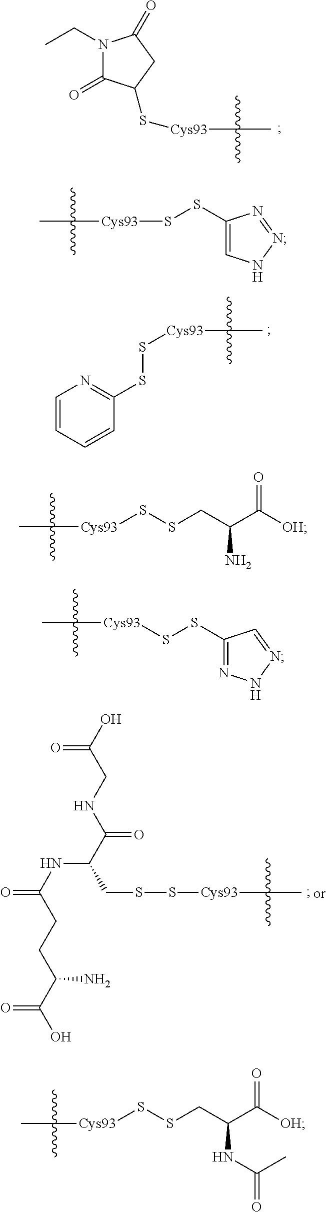

wherein the Cys93 is the β-Cys93 of the hemoglobin.

Embodiment 8. The composition of any one of Embodiments 2-7, wherein the hemoglobin comprises a terminal amino acid comprising a functionalized amine group, wherein the functionalized amine group is carbamylated, alkylated with one or more alkyl groups, carbamoylated, comprises one or more protecting groups, or a combination thereof.

Embodiment 9. The composition of any one of Embodiments 1-8, wherein the globin is a mammalian globin.

Embodiment 10. The composition of Embodiment 9, herein wherein the mammalian globin is a human, bovine, canine, equine, or porcine globin.

Embodiment 11. The composition of any one of Embodiments 1-10, further comprising a pharmaceutically acceptable carrier.

Embodiment 12. The composition of Embodiment 11, further comprising a reducing agent.

Embodiment 13. The composition of Embodiment 12, wherein the reducing agent is ascorbic acid, N-acetylcysteine, sodium dithionite, methylene blue, glutathione, B5/B5-reductase/NADH, or a combination thereof.

Embodiment 14. The composition of any one of Embodiments 1-13, wherein the composition is de-oxygenated.

Embodiment 15. An isolated hemoglobin comprising a β-Cys93 that is covalently modified to inhibit one or both salt bridges between β-Asp94, β-His146 and α-Lys40.

Embodiment 16. The isolated hemoglobin of Embodiment 15, wherein the β-Cys93 is covalently modified to have a structure satisfying any one or more of the following formulas:

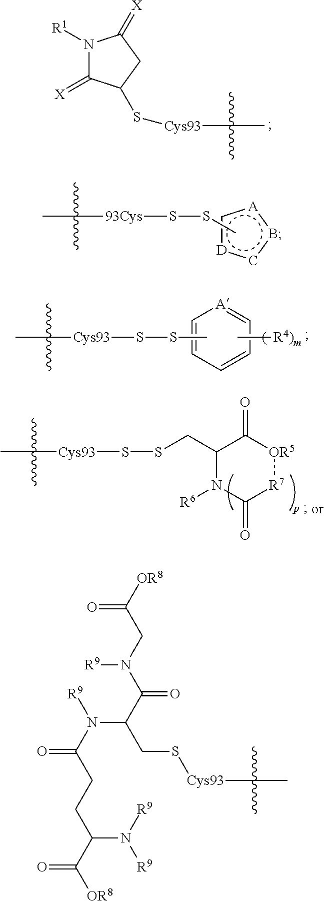

wherein

each X independently is selected from oxygen, sulfur, NR, or CRR′, wherein each R and R′ independently is hydrogen, aliphatic, heteroaliphatic, haloaliphatic, haloheteroaliphatic, aromatic, or a combination thereof;

R¹ is hydrogen, aliphatic, heteroaliphatic, haloaliphatic, haloheteroaliphatic, aromatic, or a combination thereof;

each of A, B, C, and D independently is C, CR³, N, NR², or O, wherein each of R² and R³ independently is hydrogen, aliphatic, heteroaliphatic, haloaliphatic, haloheteroaliphatic, aromatic, or a combination thereof;

A′ is N, CR⁴, or CH;

each R⁴ independently is aliphatic, heteroaliphatic, aromatic, an organic functional group, or any combination thereof;

m is an integer ranging from 0 to 5;

each of R⁵, R⁶, and R⁷ independently is hydrogen, aliphatic, heteroaliphatic, haloaliphatic, haloheteroaliphatic, aromatic, or a combination thereof;

the dotted line indicates an optional bond between the illustrated oxygen atom and the R⁷ group;

p can be 1 or 0 and when p is 0, the nitrogen atom is further bound to a second R⁶ group, which can be the same or different from the other R⁶ group;

each R⁸ independently is hydrogen, aliphatic, heteroaliphatic, haloaliphatic, haloheteroaliphatic, aromatic, or a combination thereof;

each R⁹ independently is hydrogen, aliphatic, heteroaliphatic, haloaliphatic, haloheteroaliphatic, aromatic, or a combination thereof; and

wherein the Cys93 is the β-Cys93 of the hemoglobin.

Embodiment 17. The isolated hemoglobin of Embodiment 15 or Embodiment 16, wherein the β-Cys93 is covalently modified to have a structure selected from

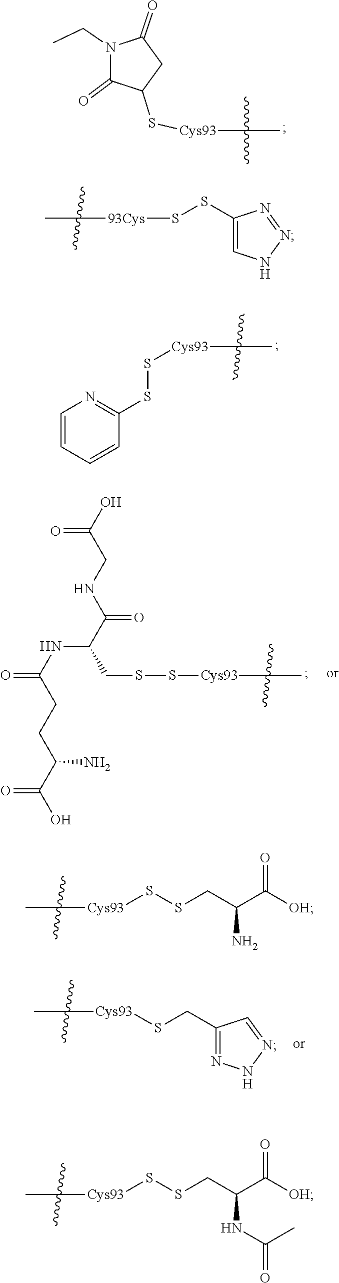

wherein the Cys93 is the β-Cys93 of the hemoglobin.

Embodiment 18. The isolated hemoglobin of any one of Embodiments 15-17, wherein the hemoglobin comprises a terminal amino acid comprising a functionalized amine group, wherein the functionalized amine group is carbamylated, alkylated with one or more alkyl groups, carbamoylated, comprises one or more protecting groups, or a combination thereof.

Embodiment 19. The isolated hemoglobin of any one of Embodiments 15-18, wherein the hemoglobin is a mammalian hemoglobin.

Embodiment 20. The isolated hemoglobin of Embodiment 19, wherein the mammalian hemoglobin is a human, bovine, canine, equine, or porcine hemoglobin.

Embodiment 21. A method of treating carboxyhemoglobinemia in a subject, comprising:

selecting a subject with carboxyhemoglobinemia; and

administering to the subject a therapeutically effective amount of the composition of any one of Embodiments 1-14.

Embodiment 22. A method of removing carbon monoxide from hemoglobin in blood or animal tissue, comprising contacting the blood or animal tissue with a composition of any one of claims 1-14, thereby removing carbon monoxide from hemoglobin in the blood or animal tissue.

Embodiment 23. The method of Embodiment 22, wherein the blood or animal tissue is in a subject, and wherein contacting the blood or animal tissue with the composition comprises administering a therapeutically effective amount of the composition to a subject.

Embodiment 24. The method of any one of Embodiments 21-23, wherein the subject is human, and the globin is human myoglobin or human hemoglobin.

Embodiment 25. The method of any one of Embodiments 22-24, comprising selecting a subject with carboxyhemoglobinemia prior to administering the composition to the subject.

Embodiment 26. The method of any one of Embodiments 21 and 23-25, wherein the subject has at least 5%, at least 10%, at least 15%, at least 20%, at least 30%, at least 40% or at least 50% carboxyhemoglobin in their blood.

Embodiment 27. The method of any one of Embodiments 21 and 23-26, wherein the composition is administered intravenously or intramuscularly.

Embodiment 28. The method of any one of Embodiments 21 and 23-27, wherein the composition is administered by intravenous infusion, intraperitoneal injection or intramuscular injection.

Embodiment 29. A method of preparing an isolated, modified hemoglobin for therapeutic use, comprising:

isolating hemoglobin from whole blood, packed red blood cells, or a combination thereof; reacting the hemoglobin with a reactant having a structure satisfying any one or more of Formulas I-V to break one or more disulfide bridges and form hemoglobin which is covalently modified at β-Cys93; and

isolating the hemoglobin which is covalently modified at β-Cys93;

wherein Formulas I-V are

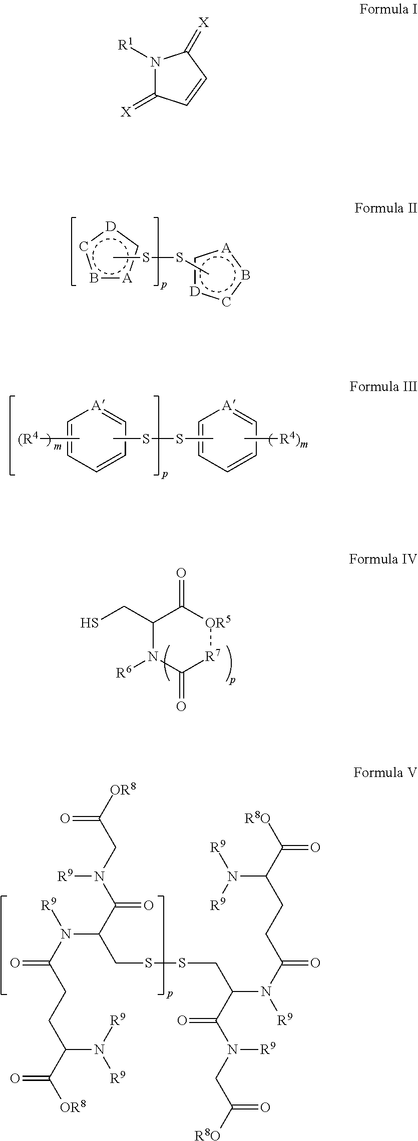

wherein

each X independently is selected from oxygen, sulfur, NR, or CRR′, wherein each R and R′ independently is hydrogen, aliphatic, heteroaliphatic, haloaliphatic, haloheteroaliphatic, aromatic, or a combination thereof;

R¹ is hydrogen, aliphatic, heteroaliphatic, haloaliphatic, haloheteroaliphatic, aromatic, or a combination thereof;

each of A, B, C, and D independently is C, CR³, N, NR², or O, wherein each of R² and R³ independently is hydrogen, aliphatic, heteroaliphatic, haloaliphatic, haloheteroaliphatic, aromatic, or a combination thereof;

A′ is N, CR⁴, or CH;

each R⁴ independently is aliphatic, heteroaliphatic, aromatic, an organic functional group, or any combination thereof;

m is an integer ranging from 0 to 5;

each of R⁵, R⁶, and R⁷ independently is hydrogen, aliphatic, heteroaliphatic, haloaliphatic, haloheteroaliphatic, aromatic, or a combination thereof;

the dotted line indicates an optional bond between the illustrated oxygen atom and the R⁷ group;

each p is 1 or 0 and, for Formula IV, when p is 0, the nitrogen atom is further bound to a second R⁶ group, which can be the same or different from the other R⁶ group;

each R⁸ independently is hydrogen, aliphatic, heteroaliphatic, haloaliphatic, haloheteroaliphatic, aromatic, or a combination thereof;

each R⁹ independently is hydrogen, aliphatic, heteroaliphatic, haloaliphatic, haloheteroaliphatic, aromatic, or a combination thereof; and

wherein the Cys93 is the β-Cys93 of the hemoglobin.

Embodiment 30. The method of Embodiment 29, wherein the reactant is selected from 2,2′-dithiopyridine, 4-4′-di(1,2,3-triazole) disulfide hydrate, N-ethylmaleimide, N-acetylcysteine, cysteine, glutathione, 3-mercapto-1,2,3-triazole, 2-mercapto-pyridyl, or any combination thereof.

Embodiment 31. The method of Embodiment 29 or 30, further comprising reacting the hemoglobin, which is covalently modified at β-Cys93, with a reducing agent.

Embodiment 32. The method of any one of Embodiments 29-31, further comprising placing the hemoglobin which is covalently modified at β-Cys93 in an oxygen free environment.

Embodiment 33. The method of any one of Embodiments 29-32, wherein the whole blood or packed red blood cells are human, porcine, canine, equine or bovine.

The following examples are provided to illustrate certain particular features and/or embodiments. These examples should not be construed to limit the disclosure to the particular features or embodiments described.

### EXAMPLES

### Example 1

### CO Scavenging Rapidly Removes CO-Hb in CO Poisoned Mice In Vivo

Mice were exposed to air with 1500 ppm CO gas for an average of 50 minutes, causing CO-Hb levels to increase to 64% +/−1%. Prior to exposure, mice were surgically instrumented with placement of femoral artery and vein catheters for blood pressure monitoring, blood sampling and infusions of recombinant neuroglobin (rNgb)—another type of CO scavenging globin protein—or PBS (control). 250 μL of 8-12 mM rNgb or PBS was infused within 4 minutes using a Harvard infusion pump Immediately after infusion and every 5 minutes, 5 μL of blood was collected for measurement of CO-Hb. After 5 minutes of return to normal air, the CO-Hb levels dropped by an average of 32.8% in the group that received rNgb versus 13.3% in the group that received PBS

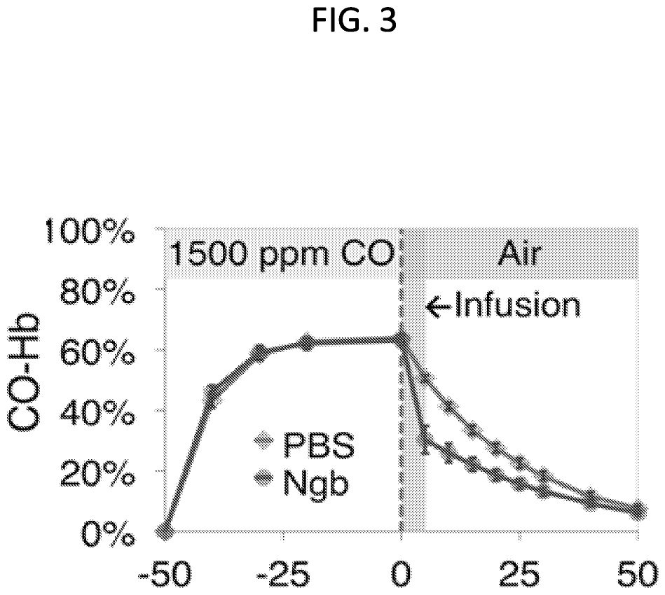

(FIG. 3). After 60 minutes, the mice were sacrificed and the urinary bladder contained mM concentrations of rNgb. It was shown that rNgb acts as an CO chelator in vivo, quickly reducing CO-Hb levels, and is filtered through the kidneys.

### Example 2

### Measuring the Ability of CO Scavenging Agents to Reverse CO Induced Mitochondrial Inhibition

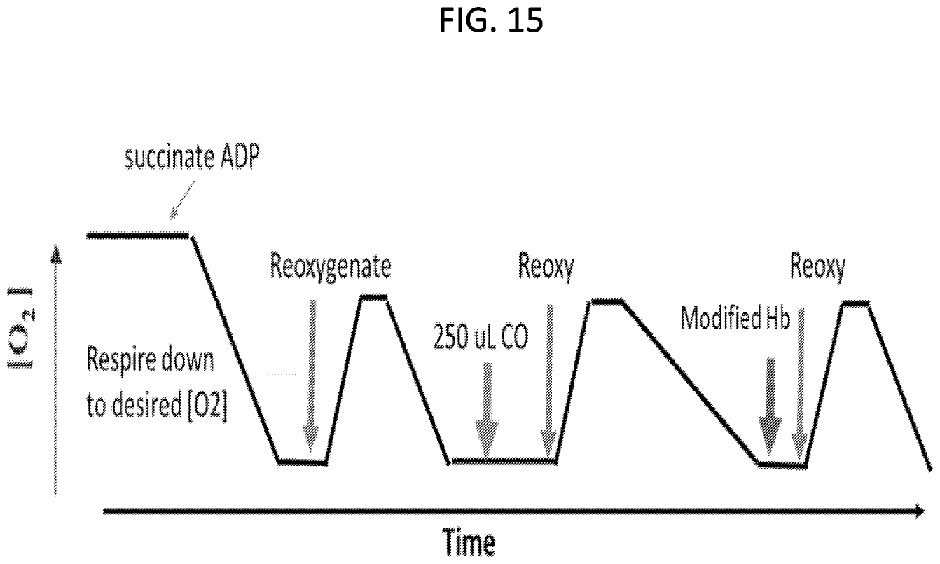

Mitochondrial respiration was measured before and after CO gas exposure in a Clark-type oxygen electrode respirometry system. The effects of infusion of both reduced hemoglobin and myoglobin were demonstrated. Fresh liver was collected in a normal rat, and mitochondria were isolated through differential centrifugation. For liver tissue, fresh liver was collected in a normal rat and then homogenized. The resulting mitochondria and liver tissue was put into the Clark-type electrode air tight reaction chamber, then substrates (succinate (mitochondria) or malate and pyruvate (liver) and ADP) were added (FIG. 15). Mitochondria then respired to 0% oxygen and then the system was reoxygenated with a pipetted injection of room air. Mitochondria respired back down to desired O₂ concentration. At this point, CO was added, either in gas form or saturated PBS solution. The system was then reoxygenated, and respiration occurred down to 0%. These rates of respiration were compared with pre-CO exposure. The reason for the first reoxygenation step is to more equally compare rates of mitochondria that have experienced some hypoxia, which can damage their function. After this was completed, CO scavenging agents were added, the system was reoxygenated and this final rate of respiration was compared both to pre-CO and post-CO respiration.

### Example 3

### In Vitro CO Scavenging from Red Blood Cells Encapsulated HbCO with Stripped Hemoglobin and NEM-Hb

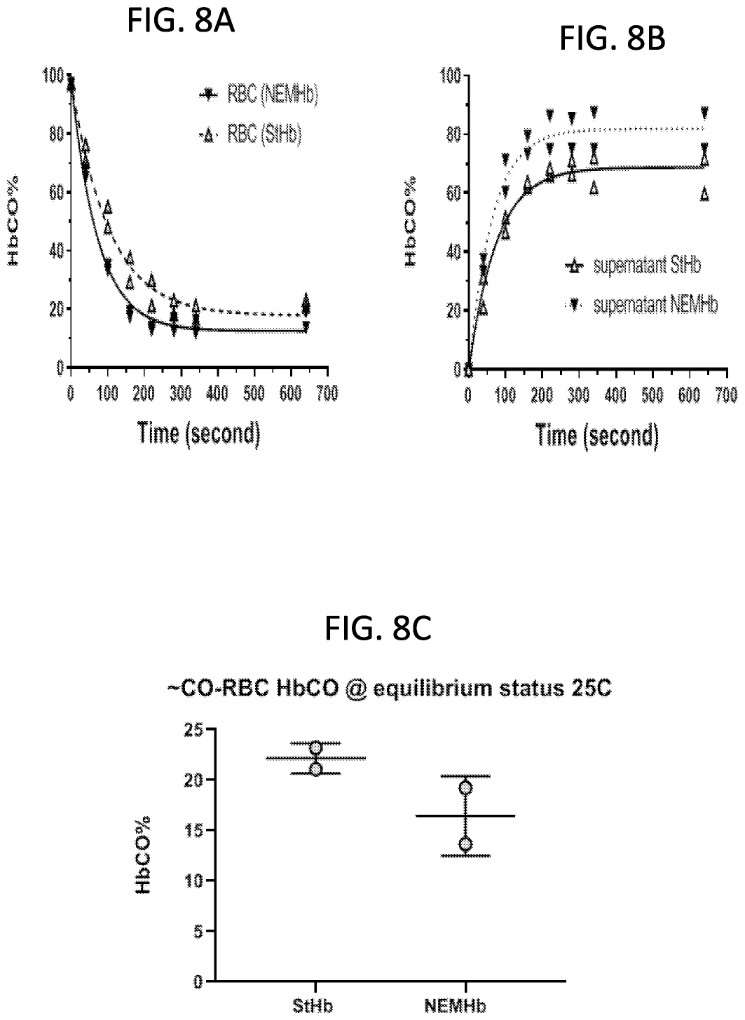

CO transfer from red blood cells to hemoproteins was started by incubation of CO saturated red blood cells (with 100% HbCO state Hb) with free StHb and NEMHb in anaerobic conditions with the presence of dithionite at 25° C. (FIGS. 8A and 8B). The ratio of heme in CO-red blood cells to hemoproteins was 1:1. The half-life of HbCO conversion to deoxyHb in the red blood cells was decreased to 71.35 seconds and 50.25 seconds, respectively in StHb and NEMHb from greater than a 200 minute half-life for the control of PBS. The hemoprotein molecules reached an equilibrium with the HbCO complex eventually, where the half-life conversion of HbCO to deoxyHb returned to normal levels. This point of HbCO in equilibrium status was 22.2% for stripped Hb and 16.5% for NEMHb (FIG. 8C). The in vitro studies demonstrated that hemoproteins can remove CO from the CO-red blood cells. Additionally, these data show superior scavenging for these specifically modified, stripped hemoglobin molecules.

### Example 4

### The Ability of Specifically Modified 2,3-DPG Free Hemoglobin Molecules to Reverse Hemodynamic Collapse and Improve Survival in a Severe CO Poisoning Mouse Model

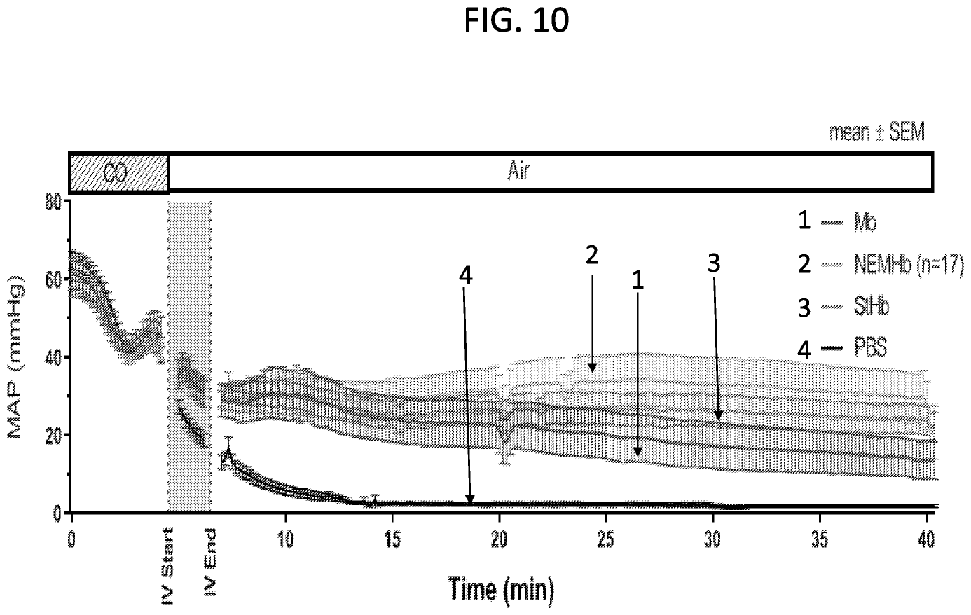

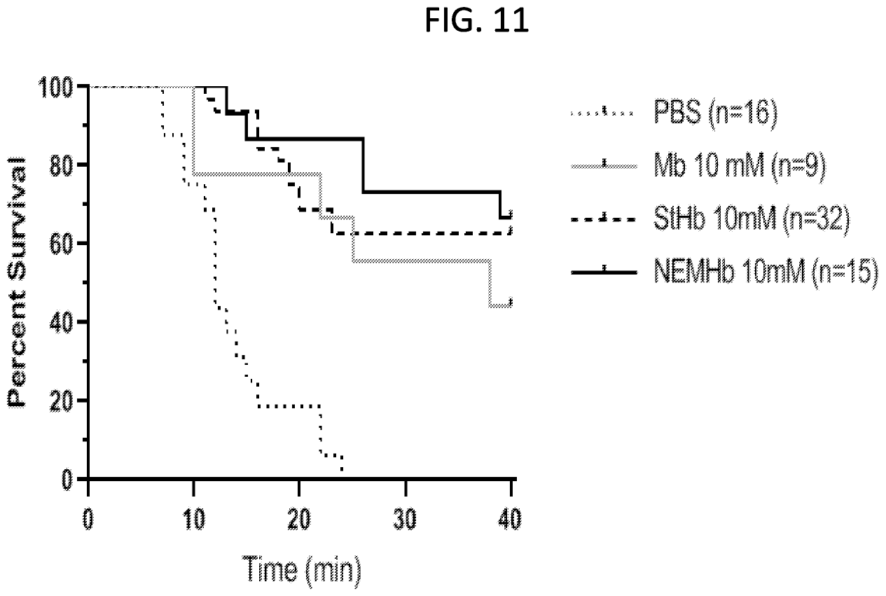

To establish a model for cardiovascular and mortality end points, tracheally intubated, ventilated, anesthetized mice were exposed to 30,000 ppm (3%) CO gas, with 21% oxygen and 1.5% isoflurane for 3 minutes and 20 seconds. Mice are surgically instrumented with placement of jugular venous (for infusion of drug) and carotid arterial (for blood pressure and heart rate monitoring) catheters. In a proof of concept model, 100% mortality was found in a group infused with 10 mL/kg of PBS post-exposure (FIG. 11). NEMHb and StHb (and to a lesser extent, Mb) partially restored the MAP, while there was a persistent hypotension and eventually death in all animals in the control group (FIGS. 10 and 11).

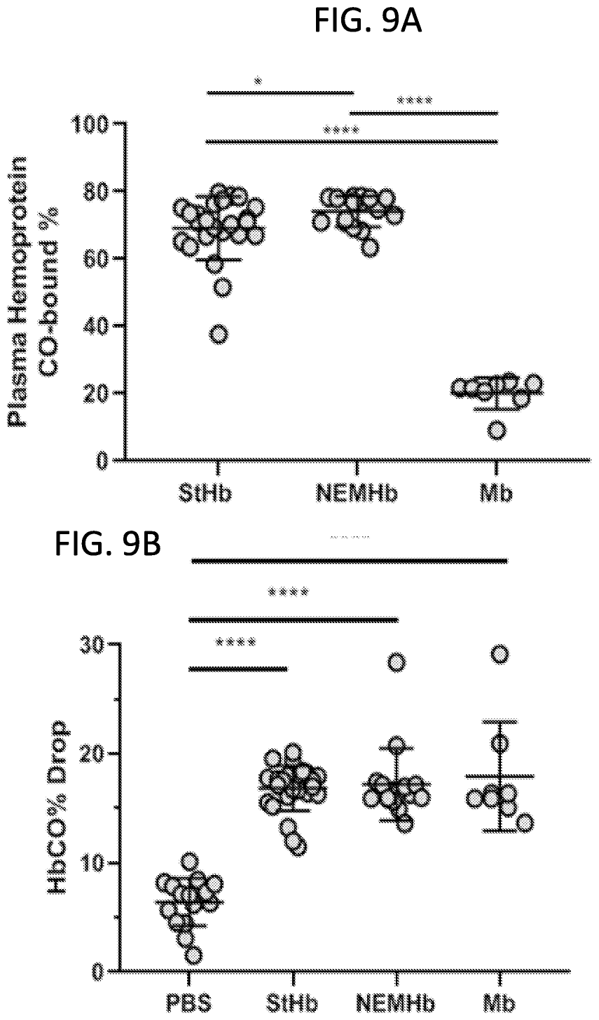

Through the jugular venous catheter, the HbCO level was sampled using spectrometry. Immediately after 3 minutes and 20 seconds of CO exposure, the HbCO level was on average 93-97%. Plasma hemoprotein concentration reached 2.0±0.3, 2.1±0.6 and 1.6±0.2 mM with the CO-bound proportion of 69.9±10.6 and 74.1±4.6% for StHb and NEMHb respectively, no difference found between hemoproteins (FIGS. 9A-9B).

Hemoproteins decreased the HbCO significantly for 16.9±2.1%, 17.2±3.3%, 17.9±5.0% respectively in StHb, NEMHb, and Mb compared to 6.4±2.2% in PBS control (P<0.0001) immediately after infusion (FIG. 9). Survival was increased from zero in PBS to 62.5%, 66.7% and 44.4% respectively by StHb, NEMHb and Mb (P<0.0001) (FIG. 11).

### Example 5

### The Ability of Specifically Modified 2,3-DPG Free Hemoglobin Molecules to Reverse Decreases in Blood Pressure and Bind to HbCO in a Moderate CO Poisoning Mouse Model

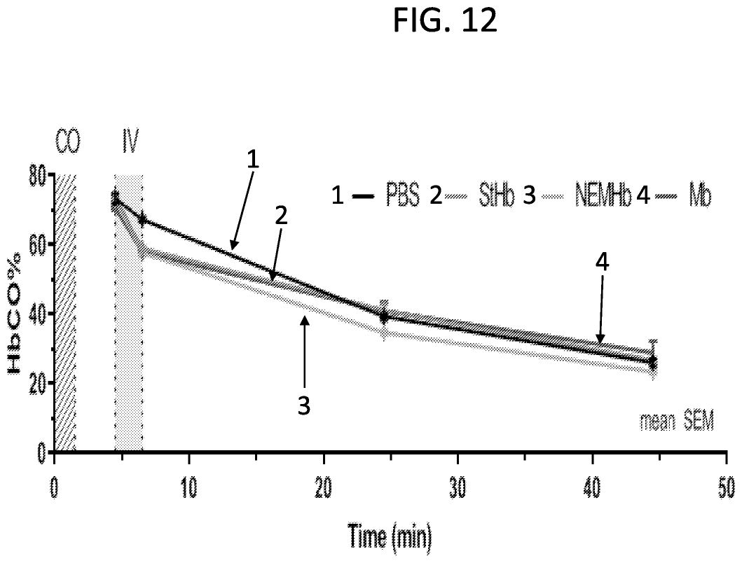

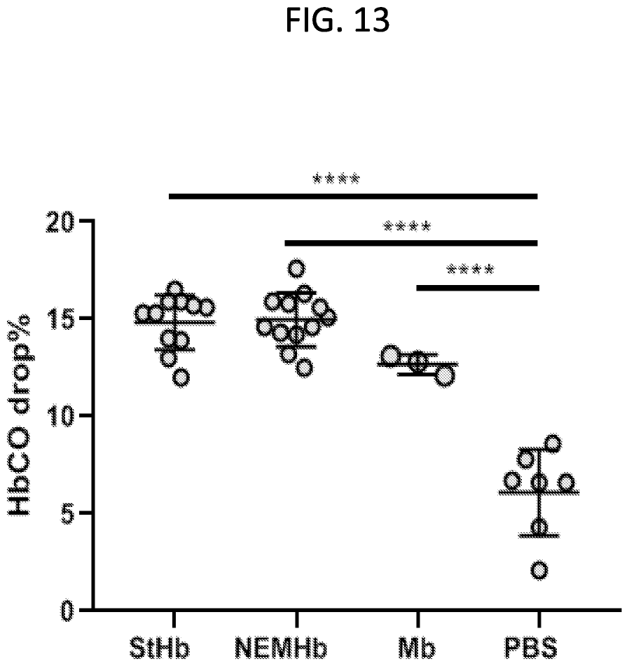

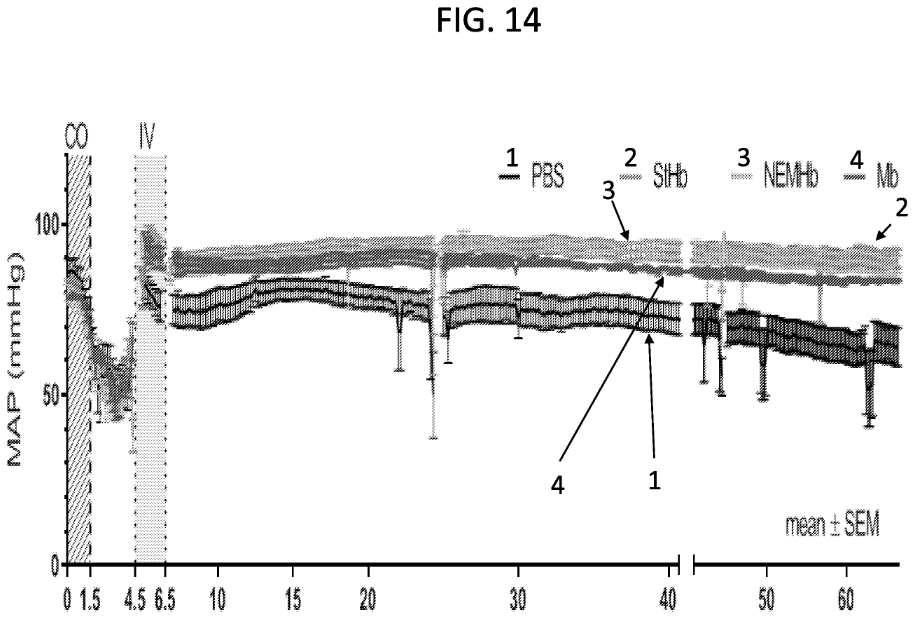

In order to further understand the hemoproteins' effects in a milder CO poisoning condition, a milder CO poisoning model was established. The tests were carried out with the body temperature maintained at 37° C. and the CO inhalation duration was shortened from 4.5 mM in the severe model to 1.5 min. StHb (n=11), NEMHb (n=12), Mb (n=3) and PBS (n=7) were compared in this model. The HbCO increased to the level of 73.0±2.5% (no difference between groups, p >0.05) and induced a hypotension, no mortality was found in the observation. There was no difference in plasm concentration (1.3±0.2 mM, 1.3±0.3 mM and 1.1±0.0 mM for StHb, NEMHb and Mb respectively, P>0.05) and CO-bound proportion (61.0±3.3%, 58.1±3.5% and *Mb-HbC₀%* for StHb, NEMHb and Mb respectively, P>0.05) between hemoproteins (FIG. 12). Hemoproteins decreased the HbCO significantly for 11.8±1.4%, 15.0±1.4%, 12.7±0.5% respectively in StHb, NEMHb, and Mb compared to 6.1±2.2% in PBS as the control (P<0.0001) (FIG. 13). Both NEMHb and StHb restored the MAP and maintained it to the pre-poisoning baseline level of 89.5 mmHg and 89.2 mmHg respectively (p<0.05). While there was a persistent hypotension in the control group that the MAP was decreased for 21.6 mmHg from 86.0 mmHg (FIG. 14).

### Example 6

### Measuring the Safety of Specifically Modified 2,3-DPG Hemoglobin Molecules in Healthy Mice

Mice are infused with 10 mM of agent in a volume of 10 mL/kg (or PBS as a control). Procedures are as follows:

Inhalational anesthesia: Mice are exposed, via a mask, to 4% isoflurane for induction, then maintained on 1.5-2.0% isoflurane for the duration of surgery and drug infusion. Intravenous catheter procedure: Chlorhexidine surgical scrub is applied on the tail, followed by 70% alcohol, repeated three times. The 23 g tail vein catheter (Braintree Scientific, Inc.) is primed with normal saline and connected to a 1 ml syringe. A skin incision of 2-3 mm is made above the lateral or dorsal tail vein in the middle of the tail, the catheter inserted into the vein to the depth of 0.5 cm and secured to the vessel.

Drug administration procedure: Drugs are administered with a slow intravenous infusion (a course of 30 minutes by a pump) through the implanted tail vein catheter. The max volume for slow intravenous infusion is 25 ml/kg for a mouse (Diehl, Karl-Heinz, Robin Hull, David Morton, Rudolf Pfister, Yvon Rabemampianina, David Smith, Jean-Marc Vidal, and Cor Van De Vorstenbosch. “A good practice guide to the administration of substances and removal of blood, including routes and volumes.” Journal of Applied Toxicology: An International Journal 21, no. 1 (2001): 15-23.)) Mice are under 1.5-2.0% isoflurane throughout the infusion period to avoid discomfort.

Recovery: The catheter is removed when the infusion is completed. The vein is ligated to prevent bleeding. One drop of mixture of lidocaine and bupivacaine is placed in the incision and the 3 mm incision is closed by suture with a 6-0 surgical thread. If present, the tracheal tube is extracted and the mouse is removed from the isoflurane to a warm chamber for recovery and is returned to the cage when it recovered from the anesthesia.

Observation and necropsy: Mice are observed for 48 hours for activity, daily weight and nesting activity. At 48 hours, they are sacrificed and blood collected for study.

### Example 7

### The Ability of Hemoglobin to Reverse Mitochondrial CO Toxicity

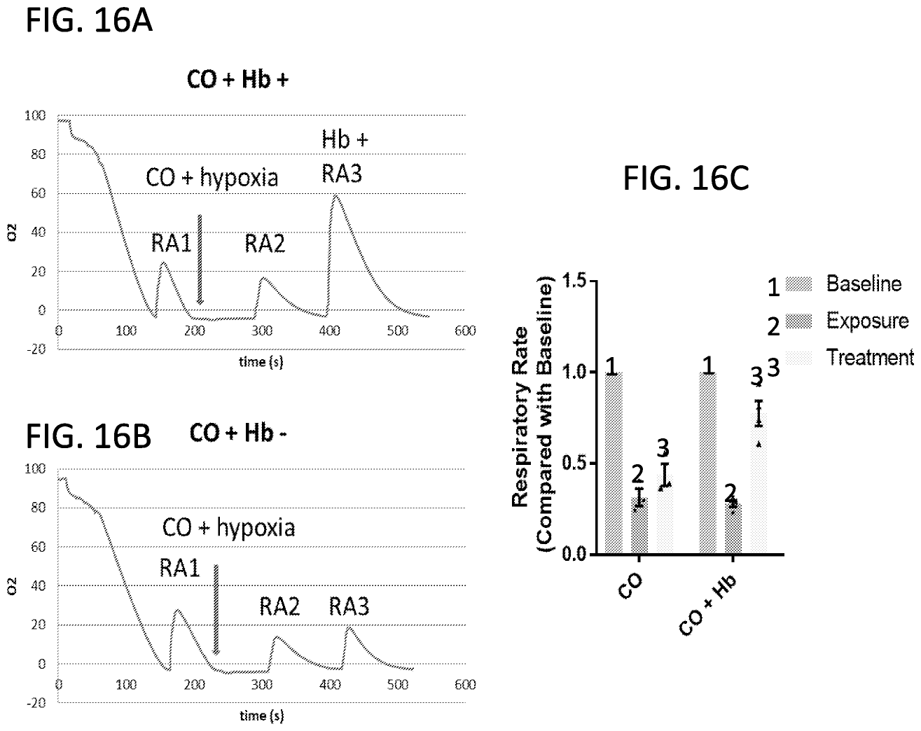

CO poisoning has long term effects on patients, and one theory is the poisoning of mitochondrial leads to generation of increased reactive oxygen species (ROS) through the inhibition of Complex IV of the electron transport chain. A model was developed to measure the amount of inhibition produced by CO exposure and quantify it through respiratory rates. In a Clark electrode, the oxygen respiration of isolated mitochondria was measured from rat livers with the addition of the substrates succinate and ADP to induce maximal respiration. The chamber was then reoxygenated with room air to obtain a baseline respiration rate. The chamber was exposed to CO saturated PBS, then maintained approximately 60 seconds in the hypoxic state to induce binding of CO to cytochrome c oxidase and the system was reoxygenated. This induces a slower observed respiration rate. It was demonstrated that CO saturated PBS induces a decrease in mitochondrial respiration in isolated mitochondria (FIG. 16B) over 2 reoxygenations. Treatment with oxy-stripped Hb prior to the last reoxygenation step recovers the respiration rate of the mitochondria (FIG. 16A) to near baseline rate (initial reoxygenation). Summary data are shown in FIG. 16C.

When also include only reoxygenation without CO exposure, and oxy-stripped Hb treatment without CO exposure, with two-way ANOVA, the interaction between CO and oxy-stripped Hb on respiration for the final reoxygenation step was highly significant (p=0.0002).

### Example 8

### Hemoglobin Based Molecules can Act as Gaseous Ligand Scavenging Molecules

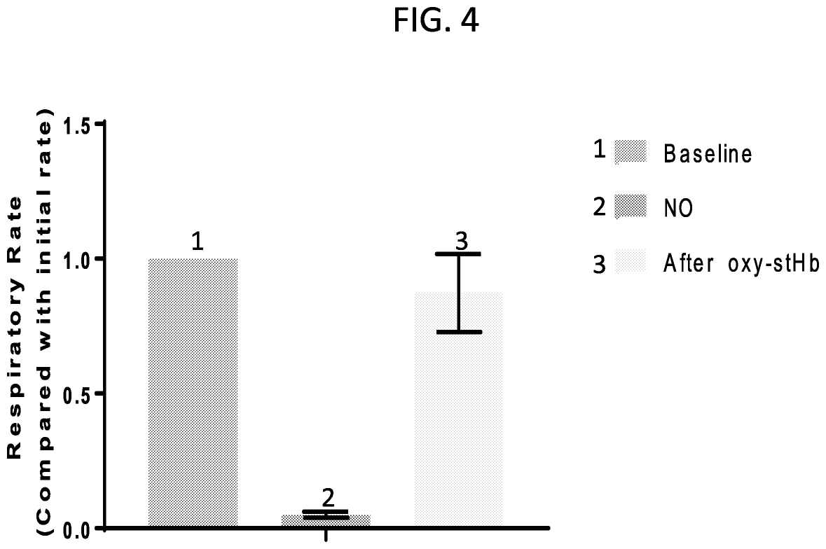

Nitric oxide is known to inhibit mitochondrial respiration, almost halting respiration altogether. This is in a manner similar to CO inhibition of mitochondrial respiration. Hemoglobin can scavenge NO and reverse the inhibition of respiration. Isolated rat liver mitochondria were placed into a Clark electrode reaction chamber. Succinate and then ADP were added for maximal respiration. Mitochondria were then exposed to Proli-NONOate, a NO donor. This halted mitochondrial respiration. With the addition of hemoglobin, respiration was restarted with the scavenging of NO (FIG. 4). The binding of CO by scavenging agent works in a similar manner (FIGS. 15 and 16A-16C).

Altogether, these results indicate that a CO scavenging agent is able to remove CO from carboxylated hemoglobin that is located inside red blood cells both in vitro and in vivo in a mouse model. In addition, hemoglobin can act as a scavenging agent for mitochondrial respiration, as demonstrated by its scavenging of NO and reversal of NO-induced inhibition.

### Example 9

### Exemplary Methods of Synthesis

In some embodiments, isolation of hemoglobin from whole blood or packed red blood cells may be carried out according to the following procedure:
1. Collect 10 mL from bag into paired black cap 50 mL Eppendorf tubes (e.g. 4, 6, or 8 tubes)
2. Dilute with 5× PBS, to total volume of 50 mL (10 mL prbc+40 mL PBS)
3. Spin 2000 g for 10 minutes, max acceleration (Allegra X-15R Centrifuge) 4° C.
4. Remove supernatant (plasma)
5. Add 40 mL PBS into same tube
6. Gently mix up and down (˜5 times)
7. Spin 2000g for 10 minutes, max acceleration (Allegra X-15R Centrifuge) 4° C.
8. Repeat steps 4-7, at least 5 times, until no color is left in supernatant
9. Remove supernatant (plasma)
10. Add 40 mL PBS into same tube
11. Gently mix up and down (˜5 times)
12. Spin 4000 g for 10 minutes, max acceleration (Allegra X-15R Centrifuge) 4° C.
13. Repeat steps 9-12, 3-5 times until no color left in supernatant
14. Remove supernatant
15. Add 30 mL of deionized water to pellet
16. Mix thoroughly by gently moving up and down
17. Incubate 1 hour at 4° C.
18. Put mixture into Oak Ridge Centrifuge Tube, PSF, size 50 mL
19. Balance out all tubes to equal weight
20. Spin at 13000 RPM on rotor JA17 for 30 minutes, max acceleration, 4° C. (Avanti J-E Centrifuge).
21. Remove supernatant and place into 50 mL Eppendorf (blue cap) in each tube.

In some embodiments, isolated hemoglobin may be modified according to the following modification protocol:
1) Into 1-5 mM stripped hemoglobin (Hb) in pH 7.4 phosphate buffered saline (PBS), add ˜7.5 equivalents (up to 37.5 mM) of either 2,2′-dithiodipyridine (2-DPS, 220.31 g/mol) on ice for 1-1.5 hours.
   e.g., A 500 mM (55 mg, 500 μL) stock solution of 2-DPS was prepared in ethanol (EtOH). This solution may be frozen in the −20°. 100 μL of 10 mM stripped Hb was diluted with 190 μL of PBS. 10 μL of the 2-DPS stock was added, and the Hb solution was gently vortexed and placed on ice for 1-1.5 hours generating 2-mercaptopyridyl Hb (2MP-Hb).
2) 2MP-Hb is gel-filtered with a G25 column in a cold room (˜4° C.). New concentration can be approximated from the volume off column and original concentration.
   e.g., 3.34 mM of 2MP-Hb in 300 μL (1 μmol) was run through a PBS saturated G25 column in the cold room, which, after collection was diluted to a final volume of 1700 μL (˜580 μM Hb).
3) Incubate 2MP-Hb in 4° C. refrigerator overnight with approximately 20-fold excess of (reduced) thiol modifying agent dissolved in PBS. Make the appropriate stock solution of the reduced thiol first (e.g. 100 mM).
   i. reduced glutathione, GSH: 307.32 g/mol, yielding GS-Hb
   ii. reduced N-acetyl-cysteine, NAC: 163.2 g/mol, yielding NAC-HB
   iii. reduced cysteine, Cys: 121.16 g/mol, yielding CysS-Hb
   e.g., 30.7 mg of reduced glutathione (0.1 mmol) was dissolved in 1 mL of PBS generating a 100 mM solution and kept on ice. To achieve a 20-fold excess (20×580 μM or 11.6 mM), 197.2 μL of the 100 mM GSH was added to the 1.7 mL of 2MP-Hb.
4) Concentrate modified Hb using 4 mL 10K cutoff centricons (or up to 50K cutoff in the 15 mL centricons if needed). Wash new centricons with nanopure water first by centrifuging at 4000 RPM for 10 minutes. Concentrated modified Hb to a volume in which G25 column(s) may be used (4000 RPM, ˜15 mM, 4° C.). Use a G25 in the cold room to remove remaining excess reduced thiol-modifying agent. Freeze, or proceed with thiol modification test to determine extent of thiolation.

In some embodiments, isolated hemoglobin may be modified according to the following modification protocol: 4,4′-di(1,2,3-triazole) disulfide hydrate (4-DTD), MW: ˜236 g/mol for dihydrate) in the same manner as 2-DPS except increase excess to 10-fold. Proceed with step 2, but then procedure is complete. Yields 4-MTri-Hb. Dissolve 11.8 mg 4-DTD into 500 μL of a 50/50 H₂O/EtOH mixture (sonication will be required, but will dissolve slowly) generating a 100 mM solution. 200 μL of the TAzS was then combined with 100 μL of 10 mM stripped Hb, gently vortexed, and placed over ice for 1.5 hours.

In some embodiments, isolated hemoglobin may be modified according to the following modification protocol:

Isolated Hemoglobin preparation for modification processing
1. Mix together isolated hemoglobin into 300 mL Falcon sterile cell culture flask
2. Sample small amount of mixed product to determine heme concentration on spectroscopy
a. Usual concentration→2.5-4 mM.
Acceptable endpoints for Isolated Hemoglobin Interim Product
Hemoglobin concentration >1.5mM by spectroscopy
Methemoglobin<5%; Oxyhemoglobin>95%.
NEM modification preparation
1. Remove isolated hemoglobin and put into new Eppendorf 50 mL tube, with enough room for a 3:1 NEM:measured heme concentration. For instance, if have 3 mM hemoglobin, mix with small volume high concentration NEM to get 9 mM end concentration NEM. Dissolve powder of NEM with 1 mL PBS.
a. If adding solution of NEM, do not exceed 100 mM of solubilized NEM
2. Incubate in room temperature for 1 hour in Eppendorf tube on a rocker
3. Load 15 mL of this mixture into a 50 mL concentrator column (Amicon ultra 15-50 KDa)
4. Spin for 4000g max accel 4° C. for 30 minutes
5. Empty effluent and collect ˜5 mL of material in collector
6. Pipette out all the NEM- Hb in a 50 ml conical tube (blue cap) then proceed to purifying through column
7. Ensure clean column with no PBS left in headspace
a. Column should be cleaned with PBS 20 ml at least 3× times prior to use. For a new column, discard the storing fluid and wash with PBS 20 ml each time at least 4× times.
8. Take 250-300 μL of stripped-Hb and NEM mixture at a time and load into G25 Sephadex column
9. Allow hemoglobin mixture to pass just below the filter, so no hemoglobin mixture is left on the surface
10. Add another 300 μL (600 μL total) of stripped-Hb and NEM mixture at a time and load into G25 Sephadex column
11. Allow hemoglobin mixture to pass just below the filter, so no hemoglobin mixture is left on the surface
12. Add 0.3 mL of PBS, allow it to pass through surface of column
13. Repeat 3× total times
14. Add 5 mL of PBS
15. Start collecting effluent when red color is eluted from column
16. Start collecting when deep pink
17. Stop collecting when deep red color ends
18. When all NEM-Hb is collected, place entire mixture into a 50 mL concentrator column (Amicon ultra 15-50 KDa)
19. Spin for 4000 g (Allegra X-15R Centrifuge) max accel 4° C. for 30 minutes
20. Empty effluent
21. Will have ˜1 mL of NEM-Hb in collector on top, fill to 15 mL with normal saline
22. Reload into centrifuge
23. Spin for 4000 g (Allegra X-15R Centrifuge) max accel 4° C. for 30 minutes
24. Repeat steps 16-19 three more times (4 total NS+NEM-Hb concentrations)
25. Check concentration of NEM-Hb with spectroscopy as well as redox state
26. Adjust concentration for goal
27. Store at −80° C. aliquots (0.85 mL into Eppendorf microtube)
28. Thaw within 30 minutes of injection, on ice.

In some embodiments, isolated hemoglobin may be reduced according to the following reduction process:

In order to make the globin molecule readily bind CO, the iron must be in the reduced Fe²+ form and not in the oxidized Fe³+ form. The oxidized form will not interact with CO and be ineffective. This is done through the addition of a reducing agent such as ascorbic acid, N-acetylcysteine, sodium dithionite, methylene blue, glutathione, or B5/B5-reductase/NADH.

### Example 10

### Exemplary Biological Testing

Kinetics of carbon monoxide saturated red blood cells mixed with hemoglobin molecules: Red cells were obtained by washing 50-100 μL of blood with PBS 5 to 7 times by centrifugation at 1000 g for 5 to 10 minutes. The washed red cells were diluted in 1 to 2 ml of PBS and deoxygenated while on ice and slowly stirred by a passing flow of argon gas for up to 1 hour. For anaerobic experiments, argon was passed briefly and an excess of sodium dithionite to Hb was added to the red cells. Carboxylated red cell-encapsulated Hb was obtained by diluting the deoxygenated red cell solution with a ratio of at least 4:1. Excess CO was removed by washing the red cells 2 times with degassed PBS (containing 5-10 mM dithionite for anaerobic experiments) by centrifugation for 5 minutes at 1000 g in degassed and septum-capped 15 mL centrifuge tubes. After washing, the red cells were resuspended to a final concentration of 100-200 μM, with an excess of sodium dithionite for anaerobic experiments. Stripped Hb and NEM-Hb were prepared as described. In some experiments, after initiating the reaction, red cells were separated from Hb to measure absorbance spectra. In this case, the reaction temperature was regulated with an Isotemp stirring hotplate and water bath combination (Fisher Scientific). Red cell-encapsulated HbCO and oxygenated or deoxygenated stripped-Hb or NEM-Hb were equilibrated to 25 or 37° C. in separate glass vials. Reaction was initiated by injecting stripped-Hb or NEM-Hb into the red cell solution for a final concentration of 40 μM of both proteins. An equivalent volume of PBS (with or without dithionite) was injected into a control sample of carboxylated red cells. Periodically, 0.5 ml of the reaction and the control sample were taken and centrifuged for 30-60 seconds at 5000 g in 1.5 mL μ-centrifuge tubes. The supernatant containing stripped-Hb or NEM-Hb was removed (5 mM sodium dithionite was added in aerobic experiments to prevent autoxidation of the protein) and stored on ice. A solution of 0.5% NP40 in PBS (always containing 5 mM sodium dithionite for anaerobic experiments and sometimes for aerobic) was added to the red cell pellet to lyse the cells. Hb absorbance in the lysed red cell solution was measured with the Cary 50 spectrophotometer in a 1 cm path length cuvette. This cycle was repeated each 1.5-5 minutes six times, giving six absorbance measurements of the Hb. The control and reaction samples were continuously stirred. The time when absorbance of hemoglobin was measured in the reaction was assumed to be the time elapsed after injection of stripped-Hb or NEM-Hb to 15 or 30 seconds after the start of centrifugation (for 30 or 60 second centrifugation durations, respectively). After the last (6th) time point was measured, absorbance of the stored supernatant samples of the reaction and control mixtures was recorded as well. In some experiments, the red cells were not separated from Hb and instead, absorbance of the whole mixture was recorded with the Integrating Sphere attachment of a Cary 100 spectrophotometer. This setup collects light scattered by the red cells, thereby providing absorbance spectra sufficiently accurate for spectral deconvolution. The procedure for these experiments was the same as that for mixing stripped-Hb or NEM-Hb with pure HbCO in the Cary 50, after preparation of carboxylated red cells.

Least Squares Deconvolution: Standard reference spectra of the oxidized (met), deoxygenated (deoxy), oxygenated (O₂) and carboxylated (CO) forms of hemoglobin (Hb), and myoglobin (Mb) were obtained. After thawing protein on ice, spectra of the oxidized form were obtained by mixing with an excess of potassium ferricyanide and passing through an Econo-Pac 10DG Desalting Column (Bio-Rad Laboratories, Hercules, Calif.). Spectra of deoxygenated species were recorded after adding an excess of sodium dithionite to the oxidized form. Spectra of the oxygenated form were recorded immediately after passing deoxygenated species through the desalting column under aerobic conditions. Spectra of the carboxylated form were measured after mixing the deoxygenated species with CO-saturated buffer in a ratio of 1:4. All standard spectra were collected at 20, 25, and 37° C. on the Cary 50 spectrophotometer. Deconvolution of experimental spectra was performed with a least-squares fitting routine in Microsoft Excel. Because the change in absorbance of the kinetic experiments is not great, all spectra composed of both Hb and Mb were always fit between 450 and 700 nm, 490 and 650 nm, and 510 and 600 nm; with and without constraining the Hb and stripped-Hb or NEM-Hb concentrations to be equal to each other, in order to confirm the accuracy of the deconvolution. For the same purpose, a parameter that could shift the spectra horizontally, along the wavelength axis, was sometimes included in the fit. Absorbance spectra from anaerobic experiments were deconvoluted using carboxylated and deoxygenated standards of Hb and stripped-Hb or NEM-Hb. Absorbance spectra from aerobic experiments were deconvoluted using the standards of the oxidized, carboxylated and oxygenated forms of Hb and Mb. For the red cell experiments where Hb was separated from stripped-Hb or NEM-Hb and dithionite was afterwards added to either red cells in aerobic experiments or to the supernatant in anaerobic experiments, deoxygenated standards were used in deconvolution instead of the oxygenated and oxidized forms. Before deconvoluting spectra collected with the Stopped-Flow spectrometer, and sometimes those with the HP8453, absorbance values were remapped to the same wavelengths as those used by the Cary 50 spectrophotometer using the interpl function of Matlab, employing piecewise cubic hermite interpolation.

### Example 11

### Blood Chemistry Following Treatment with NEM-Hb and StHb

This example describes a study to evaluate blood chemistry in animals following treatment with modified globin proteins.

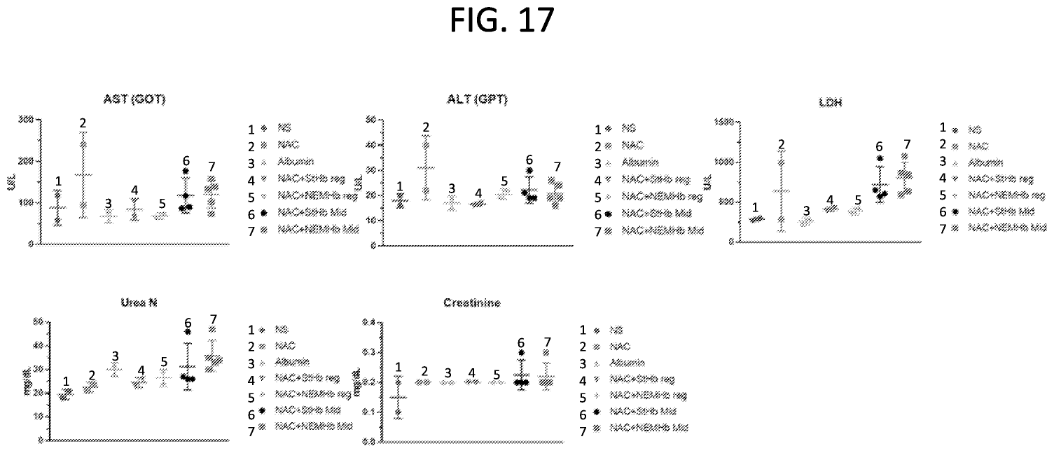

Mice were treated with either normal saline (control); 4000 mg/kg albumin (control); 100 mM N-acetyl cysteine (NAC) (control); 4 mM NEM-Hb+40 mM NAC (1600 mg/kg NEM-Hb, regular dose); 4 mM stripped Hb+40 mM NAC (1600 mg/kg stripped Hb, regular dose); 10 mM NEM-Hb+100 mM NAC (4000 mg/kg NEM-Hb, medium dose); or 10 mM stripped Hb+100mM NAC (4000 mg/kg stripped Hb, medium dose). Following treatment, plasma samples from the mice were evaluated for levels of AST, ALT, LDL, urea and creatinine (FIG. 17).

In all cases, infusion of StHb and NEM-Hb did not alter significantly the values for AST, ALT, LDL, urea and creatinine. The reported values are within normal ranges. This indicates that infusion of StHb and NAM-Hb at the indicated concentrations did not induce liver or kidney toxicity.

### Example 12

### Affinity of Modified Hemoglobin Proteins for CO

Overall affinity of StHb and NEM-Hb for CO was determined (Table 2). The value for R-state Hb is from Cooper et al. (Biochim Biophys Acta 1411(2-3): 290-309, 1999).

TABLE 2
Affinity of native and modified hemoglobin proteins for CO

| Hemoglobin | K_{A}CO (kₒₙ/k_{off} (M⁻¹) |
|---|---|
| R-state Hb | 6.0 × 10⁸ |
| St-Hb | 8.7 ± 2.9 × 10⁸ |
| NEM-Hb | 1.1 ± 0.3 × 10⁹ |

The determined parameters indicate a higher affinity of St-Hb and NEM-Hb towards CO as compared to native Hb (1.45-fold higher for St-Hb, 1.83-fold higher for NEM-Hb) indicating that CO will bind preferentially to the modified hemoglobins.

### Example 13

### Stability of StHb and NEM-Hb Using Various Excipients

This example describes a study to test the stability of StHb and NEM-Hb in the presence of different excipients. The results are shown in Tables 3A-3D below.

All assays started with a 100% reduced sample (100% oxyHb, heme iron in +2 oxidation state). The oxidized form (metHb, heme iron in +3 oxidation state) is inactive towards CO; thus the target of the formulation is to achieve the highest amount of oxyHb after the storage period. Values shown are average±standard deviation for 3 samples. Percentage of oxyHb and metHb was determined by UV-visible spectroscopy and spectral deconvolution using published methods (Azarov et al., Sci Transl Med 8(368): 368ra173, 2016; Huang et al., J Clin Invest 115(8): 2099-2107, 2005).

As shown in Tables 3A-3D, a number of formulations have been identified that can maintain more than 95% of the active forms of StHb or NEM-Hb after 1 month of storage.

TABLE 3A
Stability tests for StHb and NEM-Hb in the presence of different excipients

| | 7 days room temp | | 14 days room temp | | 14 days 4° C. | | 1 month −20° C. | |
|---|---|---|---|---|---|---|---|---|
| 10 mM StHb pH 7.4 | % OxyHb | % MetHb | % OxyHb | % MetHb | % OxyHb | % MetHb | % OxyHb | % MetHb |
| Condition 1 | 91.07 ± 1.57 | 8.93 ± 1.57 | 83.91 ± 0.20 | 16.09 ± 0.20 | 98.65 ± 0.18 | 1.35 ± 0.18 | 100.00 ± 0.00 | 0.00 ± 0.00 |
| 25 mM sodium citrate | | | | | | | | |
| 20 mM N-Acetyl-cysteine | | | | | | | | |
| 50 mM Glycine | | | | | | | | |
| Condition 2 | 89.64 ± 1.85 | 10.36 ± 1.85 | 82.01 ± 6.29 | 17.99 ± 6.29 | 98.70 ± 0.07 | 1.30 ± 0.07 | 100.00 ± 0.00 | 0.00 ± 0.00 |
| 25 mM sodium citrate | | | | | | | | |
| 20 mM N-Acetyl-cysteine | | | | | | | | |
| Condition 3 | 88.89 ± 0.42 | 11.11 ± 0.42 | — | — | — | — | 100.00 ± 0.00 | 0.00 ± 0.00 |
| 25 mM sodium citrate | | | | | | | | |
| 50 mM Glycine | | | | | | | | |
| Condition 4 | 87.40 ± 1.62 | 12.60 ± 1.62 | 84.65 ± 2.48 | | 98.50 ± 0.22 | 1.50 ± 0.22 | 98.79 ± 0.17 | 1.21 ± 0.17 |
| 25 mM sodium citrate | | | | | | | | |
| Condition 5 | 85.78 ± 2.78 | 14.22 ± 2.78 | — | — | — | — | 100.00 ± 0.00 | 0.00 ± 0.00 |
| 25 mM sodium citrate | | | | | | | | |
| 20 mM N-Acetyl-cysteine | | | | | | | | |
| 50 mM Histidine | | | | | | | | |
| Condition 6 | 91.62 ± 0.17 | 8.38 ± 0.17 | — | — | — | — | 100.00 ± 0.00 | 0.00 ± 0.00 |
| 25 mM sodium citrate | | | | | | | | |
| 50 mM Histidine | | | | | | | | |
| Condition 7 | 88.74 ± 0.69 | 11.26 ± 0.69 | 78.86 ± 1.78 | 21.14 ± 1.78 | 98.42 ± 0.09 | 1.58 ± 0.09 | 94.59 ± 0.31 | 5.41 ± 0.31 |
| 25 mM sodium citrate | | | | | | | | |
| 20 mM N-Acetyl-cysteine | | | | | | | | |
| 50 mM Glycine | | | | | | | | |
| Condition 8 | 89.61 ± 3.64 | 10.39 ± 3.64 | — | — | — | — | 100.00 ± 0.00 | 0.00 ± 0.00 |
| 25 mM sodium citrate | | | | | | | | |
| 20 mM N-Acetyl-cysteine | | | | | | | | |
| 50 mM Glycine | | | | | | | | |
| Condition 9 | 86.44 ± 3.66 | 13.56 ± 3.66 | — | — | — | — | 100.00 ± 0.00 | 0.00 ± 0.00 |
| 25 mM sodium citrate | | | | | | | | |
| 50 mM Arginine | | | | | | | | |
| 50 mM Glutamic acid | | | | | | | | |

TABLE 3B
Stability tests for StHb and NEM-Hb in the presence of different excipients

| | 7 days room temp | | 14 days room temp | | 14 days 4° C. | | 1 month −20° C. | |
|---|---|---|---|---|---|---|---|---|
| 10 mM NEMHb pH 7.4 | % OxyHb | % MetHb | % OxyHb | % MetHb | % OxyHb | % MetHb | % OxyHb | % MetHb |
| Condition 1 | 79.42 ± 0.74 | 20.58 ± 0.74 | 37.18 ± 2.89 | 62.82 ± 2.89 | 96.79 ± 0.37 | 3.21 ± 0.37 | 96.51 ± 0.12 | 3.49 ± 0.12 |
| 25 mM sodium citrate | | | | | | | | |
| 25 mM N-Acetyl-cysteine | | | | | | | | |
| 50 mM Glycine | | | | | | | | |
| Condition 2 | 73.02 ± 1.41 | 26.98 ± 1.41 | 34.09 ± 0.75 | 65.91 ± 0.75 | 96.10 ± 0.21 | 3.90 ± 0.21 | 96.89 ± 0.39 | 3.11 ± 0.39 |
| 25 mM sodium citrate | | | | | | | | |
| 25 mM N-Acetyl-cysteine | | | | | | | | |
| Condition 3 | 78.62 ± 0.44 | 21.38 ± 0.44 | 38.55 ± 0.76 | 61.45 ± 0.76 | 96.13 ± 0.61 | 3.87 ± 0.61 | 96.71 ± 0.41 | 3.29 ± 0.41 |
| 25 mM sodium citrate | | | | | | | | |
| 50 mM Glycine | | | | | | | | |
| Condition 4 | 78.00 ± 0.64 | 22.01 ± 0.64 | 39.20 ± 0.91 | 60.80 ± 0.91 | 96.87 ± 0.25 | 3.13 ± 0.25 | 95.99 ± 0.20 | 4.01 ± 0.20 |
| 25 mM sodium citrate | | | | | | | | |
| Condition 5 | 41.44 ± 1.58 | 58.56 ± 1.58 | 21.50 ± 0.66 | 78.50 ± 0.66 | 90.44 ± 3.16 | 9.53 ± 3.16 | 98.29 ± 0.14 | 1.71 ± 0.14 |
| 25 mM sodium citrate | | | | | | | | |
| 25 mM N-Acetyl-cysteine | | | | | | | | |
| 50 mM Histidine | | | | | | | | |
| Condition 6 | 64.50 ± 0.72 | 35.50 ± 0.72 | 23.39 ± 0.75 | 76.61 ± 0.75 | 94.14 ± 0.16 | 5.86 ± 0.16 | 97.74 ± 0.07 | 2.26 ± 0.07 |
| 25 mM sodium citrate | | | | | | | | |
| 50 mM Histidine | | | | | | | | |
| Condition 7 | 69.01 ± 0.51 | 30.99 ± 0.51 | 25.82 ± 3.43 | 74.18 ± 3.43 | 94.97 ± 0.23 | 5.03 ± 0.23 | 91.31 ± 0.10 | 8.69 ± 0.10 |
| No excipients | | | | | | | | |

TABLE 3C
Stability tests for StHb and NEM-Hb in the presence of different excipients

| | 7 days room temp | | 14 days room temp | | 14 days 4° C. | | 1 month −20° C. | |
|---|---|---|---|---|---|---|---|---|
| 10 mM StHb pH 7.4 | % OxyHb | % MetHb | % OxyHb | % MetHb | % OxyHb | % MetHb | % OxyHb | % MetHb |
| Condition I | 98.41 ± 1.38 | 1.59 ± 1.38 | 90.42 ± 2.93 | 9.51 ± 2.90 | 100.00 ± 0.00 | 0.00 ± 0.00 | 100.00 ± 0.00 | 0.00 ± 0.00 |
| 25 mM sodium citrate | | | | | | | | |
| 25 mM N-Acetyl-cysteine | | | | | | | | |
| 50 mM Glycine | | | | | | | | |
| 50 mg/ml Sorbitol | | | | | | | | |
| Condition II | 94.93 ± 1.01 | 5.06 ± 1.00 | 86.50 ± 1.25 | 13.50 ± 1.25 | 98.64 ± 1.54 | 1.36 ± 1.54 | 100.00 ± 0.00 | 0.00 ± 0.00 |
| 25 mM sodium citrate | | | | | | | | |
| 25 mM N-Acetyl-cysteine | | | | | | | | |
| 50 mM Glycine | | | | | | | | |
| 50 mg/ml Maltose | | | | | | | | |
| Condition III | 98.00 ± 2.04 | 1.99 ± 2.05 | 88.62 ± 3.14 | 11.38 ± 3.14 | 99.79 ± 0.22 | 0.21 ± 0.22 | 100.00 ± 0.00 | 0.00 ± 0.00 |
| 50 mg/ml Sorbitol | | | | | | | | |
| Condition IV | 92.55 ± 0.90 | 7.45 ± 0.90 | 82.64 ± 4.05 | 17.36 ± 4.05 | 99.06 ± 0.23 | 0.94 ± 0.23 | 100.00 ± 0.00 | 0.00 ± 0.00 |
| 50 mg/ml Maltose | | | | | | | | |
| Condition V | 99.82 ± 0.31 | 0.18 ± 0.31 | 88.43 ± 8.01 | 11.57 ± 8.01 | 100.00 ± 0.00 | 0.00 ± 0.00 | 100.00 ± 0.00 | 0.00 ± 0.00 |
| 25 mM sodium citrate | | | | | | | | |
| 50 mM Histidine | | | | | | | | |
| 50 mg/ml Sorbitol | | | | | | | | |
| Condition VI | 90.89 ± 3.39 | 9.11 ± 3.39 | 76.32 ± 7.42 | 23.68 ± 7.42 | 99.77 ± 0.25 | 0.23 ± 0.25 | 100.00 ± 0.00 | 0.00 ± 0.00 |
| 25 mM sodium citrate | | | | | | | | |
| 50 mM Histidine | | | | | | | | |
| 50 mg/ml Maltose | | | | | | | | |

TABLE 3D
Stability tests for StHb and NEM-Hb in the presence of different excipients

| | 7 days room temp | | 14 days room temp | | 14 days 4° C. | | 1 month −20° C. | |
|---|---|---|---|---|---|---|---|---|
| 10 mM NEM-Hb pH 7.4 | % OxyHb | % MetHb | % OxyHb | % MetHb | % OxyHb | % MetHb | % OxyHb | % MetHb |
| Condition I | 81.78 ± 1.40 | 18.22 ± 1.40 | 55.51 ± 3.86 | 44.50 ± 3.86 | 97.21 ± 0.83 | 2.80 ± 0.83 | 97.02 ± 0.08 | 2.98 ± 0.08 |
| 25 mM sodium citrate | | | | | | | | |
| 25 mM N-Acetyl-cysteine | | | | | | | | |
| 50 mM Glycine | | | | | | | | |
| 50 mg/ml Sorbitol | | | | | | | | |
| Condition II | 82.90 ± 0.23 | 17.10 ± 0.23 | 37.53 ± 0.95 | 62.47 ± 0.95 | 96.68 ± 0.46 | 3.32 ± 0.46 | 97.04 ± 0.02 | 2.96 ± 0.02 |
| 25 mM sodium citrate | | | | | | | | |
| 25 mM N-Acetyl-cysteine | | | | | | | | |
| 50 mM Glycine | | | | | | | | |
| 50 mg/ml Maltose | | | | | | | | |
| Condition III | 79.73 ± 0.78 | 20.27 ± 0.78 | 52.54 ± 4.99 | 47.46 ± 4.99 | 95.42 ± 0.42 | 4.58 ± 0.42 | 97.28 ± 0.32 | 2.72 ± 0.32 |
| 50 mg/ml Sorbitol | | | | | | | | |
| Condition IV | 76.71 ± 0.79 | 23.29 ± 0.79 | 44.05 ± 0.61 | 55.95 ± 0.60 | 94.91 ± 0.13 | 5.09 ± 0.13 | 97.04 ± 0.01 | 2.96 ± 0.01 |
| 50 mg/ml Maltose | | | | | | | | |
| Condition V | 77.64 ± 2.30 | 22.36 ± 2.31 | 35.87 ± 1.91 | 64.13 ± 1.91 | 93.86 ± 1.03 | 6.14 ± 1.03 | 96.91 ± 0.06 | 3.09 ± 0.06 |
| 25 mM sodium citrate | | | | | | | | |
| 50 mM Histidine | | | | | | | | |
| 50 mg/ml Sorbitol | | | | | | | | |
| Condition VI | 74.30 ± 0.79 | 25.70 ± 0.79 | 32.44 ± 0.76 | 67.56 ± 0.76 | 94.52 ± 0.77 | 5.48 ± 0.77 | 97.91 ± 1.81 | 2.09 ± 1.81 |
| 25 mM sodium citrate | | | | | | | | |
| 50 mM Histidine | | | | | | | | |
| 50 mg/ml Maltose | | | | | | | | |

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. A composition comprising a globin in a relaxed state, wherein at least 85% of the globin is in the relaxed state.

2. The composition of claim 1, wherein the globin is myoglobin or hemoglobin.

3. The composition of claim 2, wherein the globin is hemoglobin.

4. The composition of claim 3, wherein the hemoglobin is substantially free of 2,3-diphosphoglycerate.

5. The composition of claim 3, wherein the hemoglobin comprises a β-Cys93 that is covalently modified to inhibit one or both salt bridges between β-Asp94, β-His146 and α-Lys40.

6. (canceled)

7. (canceled)

8. The composition of claim 3, wherein the hemoglobin comprises a terminal amino acid comprising a functionalized amine group, wherein the functionalized amine group is carbamylated, alkylated with one or more alkyl groups, carbamoylated, comprises one or more protecting groups, or a combination thereof.

9. The compositon of claim 1, wherein the globin is a mammalian globin.

10. (canceled)

11. The composition of claim 1, further comprising a pharmaceutically acceptable carrier.

12. (canceled)

13. (canceled)

14. The composition of claim 1, wherein the composition is de-oxygenated.

15. An isolated hemoglobin comprising a β-Cys93 that is covalently modified to inhibit one or both salt bridges between β-Asp94, β-His146 and α-Lys40.

16. The isolated hemoglobin of claim 15, wherein the β-Cys93 is covalently modified to have a structure satisfying any one or more of the following formulas: 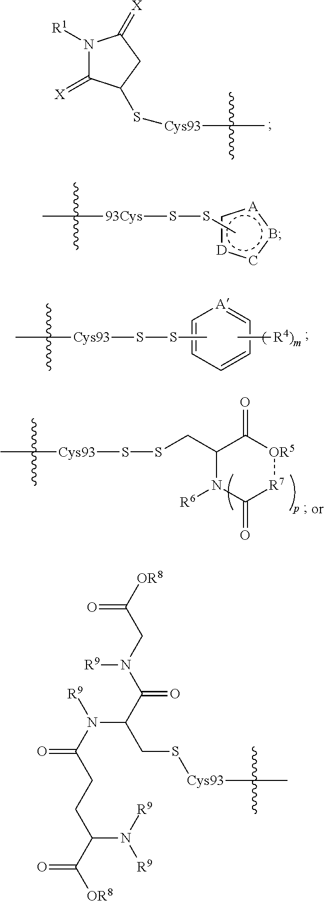
wherein
each X independently is selected from oxygen, sulfur, NR, or CRR′, wherein each R and R′ independently is hydrogen, aliphatic, heteroaliphatic, haloaliphatic, haloheteroaliphatic, aromatic, or a combination thereof;
R′ is hydrogen, aliphatic, heteroaliphatic, haloaliphatic, haloheteroaliphatic, aromatic, or a combination thereof;
each of A, B, C, and D independently is C, CR³, N, NR², or O, wherein each of R² and R³ independently is hydrogen, aliphatic, heteroaliphatic, haloaliphatic, haloheteroaliphatic, aromatic, or a combination thereof;
A′ is N, CR⁴, or CH;
each R⁴ independently is aliphatic, heteroaliphatic, aromatic, an organic functional group, or any combination thereof;
m is an integer ranging from 0 to 5;
each of R⁵, R⁶, and R⁷ independently is hydrogen, aliphatic, heteroaliphatic, haloaliphatic, haloheteroaliphatic, aromatic, or a combination thereof;
the dotted line indicates an optional bond between the illustrated oxygen atom and the R⁷ group;
p can be 1 or 0 and when p is 0, the nitrogen atom is further bound to a second R⁶ group, which can be the same or different from the other R⁶ group;
each R⁸ independently is hydrogen, aliphatic, heteroaliphatic, haloaliphatic, haloheteroaliphatic, aromatic, or a combination thereof;
each R⁹ independently is hydrogen, aliphatic, heteroaliphatic, haloaliphatic, haloheteroaliphatic, aromatic, or a combination thereof; and
wherein the Cys93 is the β-Cys93 of the hemoglobin.

17. The isolated hemoglobin of claim 15, wherein the β-Cys93 is covalently modified to have a structure selected from 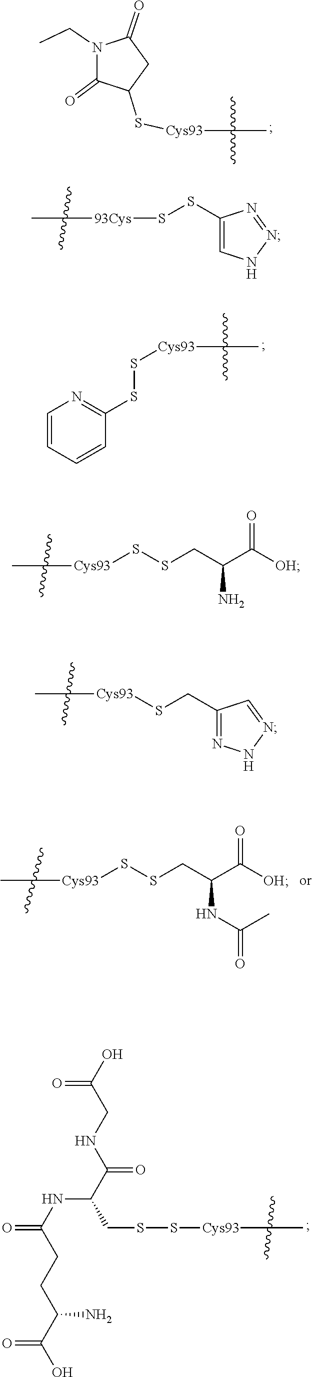
or
wherein the Cys93 is the β-Cys93 of the hemoglobin.

18. The isolated hemoglobin of claim 15, wherein the hemoglobin comprises a terminal amino acid comprising a functionalized amine group, wherein the functionalized amine group is carbamylated, alkylated with one or more alkyl groups, carbamoylated, comprises one or more protecting groups, or a combination thereof.

19. The isolated hemoglobin of claim 15, wherein the hemoglobin is a mammalian hemoglobin.

20-33. (canceled)


SEQUENCE LISTING (17 sequences)

SEQ ID NO: 1 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 2 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 3 - protein, 141 aa, Canis lupus familiaris
VLSPADKTNIKSTWDKIGGHAGDYGGEALDRTFQSFPTTKTYFPHFDLSPGSAQVKAHGKKVADALTTAVAHLDDLPGALSALSDLHAYKLRVDPVNFKLLSHCLLVTLACHHPTEFTPAVHASLDKFFAAVSTVLTSKYR

SEQ ID NO: 4 - protein, 146 aa, Canis lupus familiaris
VHLTAEEKSLVSGLWGKVNVDEVGGEALGRLLIVYPWTQRFFDSFGDLSTPDAVMSNAKVKAHGKKVLNSFSDGLKNLDNLKGTFAKLSELHCDKLHVDPENFKLLGNVLVCVLAHHFGKEFTPQVQAAYQKVVAGVANALAHKYH

SEQ ID NO: 5 - protein, 141 aa, Sus scrofa
VLSAADKANVKAAWGKVGGQAGAHGAEALERMFLGFPTTKTYFPHFNLSHGSDQVKAHGQKVADALTKAVGHLDDLPGALSALSDLHAHKLRVDPVNFKLLSHCLLVTLAAHHPDDFNPSVHASLDKFLANVSTVLTSKYR

SEQ ID NO: 6 - protein, 147 aa, sus scrofa
MVHLSAEEKEAVLGLWGKVNVDEVGGEALGRLLVVYPWTQRFFESFGDLSNADAVMGNPKVKAHGKKVLQSFSDGLKHLDNLKGTFAKLSELHCDQLHVDPENFRLLGNVIVVVLARRLGHDFNPNVQAAFQKVVAGVANALAHKYH

SEQ ID NO: 7 - protein, 142 aa, Equus caballus
MVLSAADKTNVKAAWSKVGGHAGEYGAEALERMFLGFPTTKTYFPHFDLSHGSAQVKAHGKKVGDALTLAVGHLDDLPGALSNLSDLHAHKLRVDPVNFKLLSHCLLSTLAVHLPNDFTPAVHASLDKFLSSVSTVLTSKYR

SEQ ID NO: 8 - protein, 146 aa, Equus caballus
VQLSGEEKAAVLALWDKVNEEEVGGEALGRLLVVYPWTQRFFDSFGDLSNPGAVMGNPKVKAHGKKVLHSFGEGVHHLDNLKGTFAALSELHCDKLHVDPENFRLLGNVLVVVLARHFGKDFTPELQASYQKVVAGVANALAHKYH

SEQ ID NO: 9 - protein, 142 aa, Bos taurus
MVLSAADKGNVKAAWGKVGGHAAEYGAEALERMFLSFPTTKTYFPHFDLSHGSAQVKGHGAKVAAALTKAVEHLDDLPGALSELSDLHAHKLRVDPVNFKLLSHSLLVTLASHLPSDFTPAVHASLDKFLANVSTVLTSKYR

SEQ ID NO: 10 - protein, 145 aa, Bos taurus
MLTAEEKAAVTAFWGKVKVDEVGGEALGRLLVVYPWTQRFFESFGDLSTADAVMNNPKVKAHGKKVLDSFSNGMKHLDDLKGTFAALSELHCDKLHVDPENFKLLGNVLVVVLARNFGKEFTPVLQADFQKVVAGVANALAHRYH

SEQ ID NO: 11 - protein, 142 aa, Mus musculus
MVLSGEDKSNIKAAWGKIGGHGAEYGAEALERMFASFPTTKTYFPHFDVSHGSAQVKGHGKKVADALASAAGHLDDLPGALSALSDLHAHKLRVDPVNFKLLSHCLLVTLASHHPADFTPAVHASLDKFLASVSTVLTSKYR

SEQ ID NO: 12 - protein, 147 aa, Mus musculus
MVHLTDAEKAAVSCLWGKVNSDEVGGEALGRLLVVYPWTQRYFDSFGDLSSASAIMGNAKVKAHGKKVITAFNDGLNHLDSLKGTFASLSELHCDKLHVDPENFRLLGNMIVIVLGHHLGKDFTPAAQAAFQKVVAGVATALAHKYH

SEQ ID NO: 13 - protein, 141 aa, Felis catus
VLSAADKSNVKACWGKIGSHAGEYGAEALERTFCSFPTTKTYFPHFDLSHGSAQVKAHGQKVADALTQAVAHMDDLPTAMSALSDLHAYKLRVDPVNFKFLSHCLLVTLACHHPAEFTPAVHASLDKFFSAVSTVLTSKYR

SEQ ID NO: 14 - protein, 146 aa, Felis catus
GFLTAEEKGLVNGLWGKVNVDEVGGEALGRLLVVYPWTQRFFESFGDLSSADAIMSNAKVKAHGKKVLNSFSDGLKNIDDLKGAFAKLSELHCDKLHVDPENFRLLGNVLVCVLAHHFGHDFNPQVQAAFQKVVAGVANALAHKYH

SEQ ID NO: 15 - protein, 142 aa, Macaca mulatta
MVLSPADKSNVKAAWGKVGGHAGEYGAEALERMFLSFPTTKTYFPHFDLSHGSAQVKGHGKKVADALTLAVGHVDDMPNALSALSDLHAHKLRVDPVNFKLLSHCLLVTLAAHLPAEFTPAVHASLDKFLASVSTVLTSKYR

SEQ ID NO: 16 - protein, 146 aa, Macaca mulatta
VHLTPEEKNAVTTLWGKVNVDEVGGEALGRLLLVYPWTQRFFESFGDLSSPDAVMGNPKVKAHGKKVLGAFSDGLNHLDNLKGTFAQLSELHCDKLHVDPENFKLLGNVLVCVLAHHFGKEFTPQVQAAYQKVVAGVANALAHKYH

SEQ ID NO: 17 - DNA, 646 bp, Homo sapiens
ggcatgaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggagaagtaacagggtacagtttagaatgggaaacagacgaatgatt